(19) United States
(12) Patent Application Publication
(10) Pub. No.: US 2016/0326237 A1
(43) Pub. Date: Nov. 10, 2016

(54) METHODS OF TREATMENT FOR GUILLAIN-BARRE SYNDROME

(71) Applicants: ANNEXON, INC., SOUTH SAN FRANCISCO, CA (US); THE UNIVERSITY OF GLASGOW, GLASGOW (GB)

(72) Inventors: Arnon Rosenthal, Woodside, CA (US); Michael Leviten, Palo Alto, CA (US); Hugh J. Willison, Glasgow (GB); Rhona McGonigal, Glasgow (GB)

(21) Appl. No.: 14/939,706

(22) Filed: Nov. 12, 2015

Related U.S. Application Data
(63) Continuation of application No. PCT/US2014/038267, filed on May 15, 2014.
(60) Provisional application No. 61/823,876, filed on May 15, 2013.

(51) Int. Cl. C07K 16/18

(52) U.S. Cl. CPC C07K 16/18; C07K 2317/76; C07K 2317/56; C07K 2317/34; C07K 2317/92; C07K 2317/31; A61K 2039/505

(57) ABSTRACT

This invention relates generally to methods of treatment for Guillain-Barre' Syndrome (GBS) and, more specifically, to methods involving the inhibition of the classical pathway of complement activation.

### CROSS REFERENCES TO RELATED APPLICATIONS

This application claims the benefit of U.S. Provisional Application No. 61/823,876, filed May 15, 2013, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 717192000740SeqList.txt, date recorded: May 14, 2014, size: 23 KB).

### BACKGROUND

1. Field

This invention relates generally to methods of treatment for Guillain-Barré Syndrome and more specifically to methods involving the inhibition of the classical pathway of complement activation.

2. Description of Related Art

Guillain-Barre' syndrome (GBS) represents a spectrum of acute idiopathic, usually monophasic peripheral neuropathies (Willison and Yuki, 2002; Hughes and Cornblath, 2005; van Doom et al., 2008). GBS typically presents as an ascending paralysis with weakness beginning in the feet and hands and migrating towards the trunk. As the weakness progresses upward, usually over periods of hours to days, the arms and facial muscles can also become affected. Frequently, the lower cranial nerves may be affected, leading to bulbar weakness, oropharyngeal dysphagia (drooling, or difficulty swallowing, and/or maintaining an open airway) and life-threatening respiratory difficulties. There is a mortality rate of 2-3% with most patients requiring hospitalization, and about 30% needing ventillatory assistance for treatment of respiratory failure (Burt et al. 2009). Nearly 80% of patients have a complete recovery within a few months to a year, although minor findings may persist, such as areflexia. About 5-10% of patients have one or more late relapses, in which case they are then classified as having chronic inflammatory demyelinating polyneuropathy.

Current pharmacologic therapy consists of either plasmapheresis (i.e., filtering antibodies out of the blood stream) or administration of intravenous immunoglobulins (IVIg). These two treatments are equally effective; however, a combination of the two is not significantly better than either alone. Plasmapheresis hastens recovery when used within four weeks of the onset of symptoms (Hughes et al 2003). IVIg has equivalent efficacy to plasmapheresis when started within two weeks of the onset of symptoms, and has fewer complications (Hughes et al. 2003). IVIg is usually used first because of its ease of administration and safety profile. However, the use of IVIg is not without risk; occasionally it causes hepatitis, or in rare cases, renal failure if used for longer than five days, and can also cause clotting abnormalities. Despite the availability of these therapies and improvements in supportive care, 20-30% of patients are left with some form of permanent disability, 5-10% of patients are left with severe disability (such as inability to walk unassisted), and 2-3% of patients die. Accordingly, there is a continuing need for new therapies to treat GBS.

Advances in the understanding of the immunopathogenesis of the disease have identified new targets for therapeutic intervention including the complement pathway, which is a fundamental component of the innate immune system. In brief, an aberrant immune response to myelin and/or axolemmal antigens, typically following an infection, is considered to be the fundamental cause of GBS (van Doorn et al. 2008). Human and animal model studies suggest that pathogen infection results in the production of antibodies to the pathogen gangliosides, which cross-react with host gangliosides that are abundant in neuronal cell membranes. Accumulation of such antibodies at the pre-synaptic membrane of motor neurons results in activation of the complement cascade, the formation of complement membrane attack complex (MAC), and recruitment of macrophages. The ensuing ultrastructural destruction and blockade of synaptic transmission at the neuromuscular junction can causes the muscle weakness associated with the disease (Fewou et al. 2014).

Evidence for the important role of the complement pathway in GBS comes from a variety of patient tissue studies and from animal models of GBS subtypes like Miller Fisher Syndrome (MFS). Studies of spinal roots and nerves from autopsy cases of the GBS subtype acute motor axonal neuropathy (AMAN) revealed the presence of infiltrating macrophages with extensive processes in the periaxonal space abutting the nodal and internodal axolemma and displacing the adaxonal Schwann cell membrane and myelin sheath. Approximately 50% of AMAN patients (Ho et al. 1999) and 80-90% of MFS patients (Willison and Yuki 2002) are positive for anti-ganglioside antibodies and immunocytochemistry analysis showed very intense immunoglobulin G (IgG) and complement C3d and C5b-9 (membrane attack complex [MAC]) deposits bound to the nodal and internodal axolemma in the periaxonal space. Additional studies have also shown the activation of complement products in plasma and CSF of GBS patients (Sanders et al., 1986; Hartung et al., 1987; Koski et al., 1987; Hafer-Macko et al., 1996). Yuki et al. recapitulated some of the key pathological findings in rabbits sensitized with gangliosides including GM1 (Yuki et al., 2001). In these animals, high anti-GM1 IgG antibody titres were observed, accompanied by a flaccid limb weakness. Peripheral nerves showed predominant Wallerian-like degeneration with neither lymphocytic infiltration nor demyelination, whereas IgG was deposited on ventral root axons. Macrophage infiltration into the periaxonal space and nodal complement deposits have also been demonstrated (Susuki et al., 2003). Recently, some of the molecular events that may lead to axonal conduction block have been defined, which include the disappearance of sodium channel immunoreactivity at the node of Ranvier (Susuki et al., 2007). It has also recently been shown that inhibition of MAC assembly using a monoclonal antibody inhibitor of C5 in a mouse model of MFS had a major neuroprotective effect, completely preventing any structural and functional changes at the node of Ranvier, found previously at the motor nerve terminal (Halstead et al., 2008).

Recently, clinical trials have begun in GBS for Eculizumab, which is a terminal complement inhibitor currently approved for the treatment of paroxysmal nocturnal hemoglobinuria (PNH) and atypical hemolytic uremic syndrome (aHUS) (see e.g., FDA drug label for Eculizumab). While Eculizumab was shown to provide clinical benefits in animal models, one problem with inhibiting the terminal complement pathway is that such inhibition can cause serious side-effects due to the associated weakening of a patient's immune defenses against bacterial infections. Eculizumab therapy, for example, is associated with elevated risks of meningococcal meningitis and other bacterial infections (see, e.g. FDA drug label for Eculizumab). Moreover, Eculizumab is not expected to prevent complement-dependent cell-mediated cytotoxicity (CDCC) to the extent that CDCC is driven by complement factor C3a, an anaphalotoxin produced upstream of the Eculizumab targeted C5-convertase (Klos et al. 2009).

The C1 complex is the initiating factor of the classical complement cascade, and binds directly to autoantibody complexes. C1q binding to antibody leads to the activation of the C1r and C1s enzymes leading to the production of anaphylatoxins (C3a, C4a, and C5a), and the membrane attack complex (MAC). In contrast to Eculizumab, which blocks C5a and MAC formation for all three arms of the complement system, inhibition of C1 may specifically block C5a and MAC formation that is dependent primarily upon classical pathway activators (e.g., antibodies), which would leave the lectin and alternate pathways active for fighting infection. In addition to this safety advantage for C1 inhibition, C1 blockade may be more effective inhibitor of harmful autoantibody responses because it can block MAC formation as well as the production of opsins (i.e., C3b) and inflammatory mediators (i.e., C3a, C4a) that may contribute to the pathogenesis of the disease, and which are not blocked by Eculizumab.

Accordingly, the C1 complex may be an attractive therapeutic target for GBS. Thus, there is a need to develop new treatment options for GBS patients, such as antibodies that inhibit the C1 complex and its components, which would then inhibit the early stages of complement activation, including the classical complement activation pathway.

All references cited herein, including patent applications and publications, are hereby incorporated by reference in their entirety.

### BRIEF SUMMARY

Certain aspects of the present disclosure provide anti-C1q, anti-C1s, anti-C1r, and anti-C1 complex antibodies and methods of using such antibodies for creating or preventing Guillain-Barré Syndrome (GBS) in an individual.

Certain aspects of the present disclosure are directed to methods of treating Guillain-Barre' syndrome (GBS) that include inhibiting the classical pathway of complement activation by neutralizing the complement factors C1q, C1r, or C1s, e.g., through the administration of antibodies, such as monoclonal, chimeric, humanized antibodies, antibody fragments, etc., which bind to one or more of these complement factors.

In certain aspects, the present disclosure provides a method of treating or preventing Guillain-Barré Syndrome (GBS) in an individual, comprising administering to the individual a therapeutically effective amount of an antibody, wherein the antibody is: i) an anti-C1q antibody, wherein the anti-C1q antibody inhibits the interaction between C1q and an autoantibody, or between C1q and C1r, or between C1q and C1s, or wherein the anti-C1q antibody prevents C1q from activating C1r or C1s; ii) an anti-C1r antibody, wherein the anti-C1r antibody inhibits the interaction between C1r and C1q, or between C1r and C1s, or wherein the anti-C1r antibody inhibits the catalytic activity of C1r or inhibits the processing of pro-C1r to an active protease; iii) an anti-C1s antibody, wherein the anti-C1s antibody inhibits the interaction between C1s and C1q, or between C1s and C1r, or between C1s and C2, or between C1s and C4, or wherein the anti-C1s antibody inhibits the catalytic activity of C1s or inhibits the processing of pro-C1s to an active protease; or iv) an anti-C1 complex antibody that binds to a combinatorial epitope within the C1 complex, wherein said combinatorial epitope is comprised of C1q and C1s; C1q and C1r; C1r and C1s; or C1q, C1r, and C1s; or wherein the anti-C1 complex antibody inhibits C1r or C1s activation or prevents their ability to act on C2 or C4. In other aspects, the present disclosure provides an antibody for use in treating or preventing Guillain-Barré Syndrome (GBS) in an individual, wherein the antibody is: i) an anti-C1q antibody, wherein the anti-C1q antibody inhibits the interaction between C1q and an autoantibody, or between C1q and C1r, or between C1q and C1s, or wherein the anti-C1q antibody prevents C1q from activating C1r or C1s; ii) an anti-C1r antibody, wherein the anti-C1r antibody inhibits the interaction between C1r and C1q, or between C1r and C1s, or wherein the anti-C1r antibody inhibits the catalytic activity of C1r or inhibits the processing of pro-C1r to an active protease; iii) an anti-C1s antibody, wherein the anti-C1s antibody inhibits the interaction between C1s and C1q, or between C1s and C1r, or between C1s and C2, or between C1s and C4, or wherein the anti-C1s antibody inhibits the catalytic activity of C1s or inhibits the processing of pro-C1s to an active protease; or iv) an anti-C1 complex antibody that binds to a combinatorial epitope within the C1 complex, wherein said combinatorial epitope is comprised of C1q and C1s; C1q and C1r; C1r and C1s; or C1q, C1r, and C1s; or wherein the anti-C1 complex antibody inhibits C1r or C1s activation or prevents their ability to act on C2 or C4. In other aspects, the present disclosure provides use of an antibody in the manufacture of a medicament for treating or preventing Guillain-Barré Syndrome (GBS) in an individual, wherein the antibody is: i) an anti-C1q antibody, wherein the anti-C1q antibody inhibits the interaction between C1q and an autoantibody, or between C1q and C1r, or between C1q and C1s, or wherein the anti-C1q antibody prevents C1q from activating C1r or C1s; ii) an anti-C1r antibody, wherein the anti-C1r antibody inhibits the interaction between C1r and C1q, or between C1r and C1s, or wherein the anti-C1r antibody inhibits the catalytic activity of C1r or inhibits the processing of pro-C1r to an active protease; iii) an anti-C1s antibody, wherein the anti-C1s antibody inhibits the interaction between C1s and C1q, or between C1s and C1r, or between C1s and C2, or between C1s and C4, or wherein the anti-C1s antibody inhibits the catalytic activity of C1s or inhibits the processing of pro-C1s to an active protease; or iv) an anti-C1 complex antibody that binds to a combinatorial epitope within the C1 complex, wherein said combinatorial epitope is comprised of C1q and C1s; C1q and C1r; C1r and C1s; or C1q, C1r, and C1s; or wherein the anti-C1 complex antibody inhibits C1r or C1s activation or prevents their ability to act on C2 or C4.

In other aspects, the present disclosure provides a method of treating or preventing Guillain-Barré Syndrome (GBS) in an individual, comprising administering to the individual a therapeutically effective amount of an anti-C1q antibody wherein the antibody is: i) an isolated anti-C1q antibody comprising a light chain variable domain and a heavy chain variable domain, wherein the light chain variable domain comprises the HVR-L1, HVR-L2, and HVR-L3 of the monoclonal antibody M1 produced by a hybridoma cell line with ATCC Accession Number PTA-120399 or progeny thereof and/or wherein the heavy chain variable domain comprises the HVR-H1, HVR-H2, and HVR-H3 of the monoclonal antibody M1 produced by a hybridoma cell line with ATCC Accession Number PTA-120399 or progeny thereof or ii) an isolated anti-C1q antibody which binds essentially the same C1q epitope as the antibody M1 produced by the hybridoma cell line with ATCC Accession Number PTA-120399 or anti-C1q binding fragments thereof. In other aspects, the present disclosure provides an anti-C1q antibody for use in treating or preventing Guillain-Barré Syndrome (GBS) in an individual, wherein the anti-C1q antibody is: i) an isolated anti-C1q antibody comprising a light chain variable domain and a heavy chain variable domain, wherein the light chain variable domain comprises the HVR-L1, HVR-L2, and HVR-L3 of the monoclonal antibody M1 produced by a hybridoma cell line with ATCC Accession Number PTA-120399 or progeny thereof and/or wherein the heavy chain variable domain comprises the HVR-H1, HVR-H2, and HVR-H3 of the monoclonal antibody M1 produced by a hybridoma cell line with ATCC Accession Number PTA-120399 or progeny thereof or ii) an isolated anti-C1q antibody which binds essentially the same C1q epitope as the antibody M1 produced by the hybridoma cell line with ATCC Accession Number PTA-120399 or anti-C1q binding fragments thereof. In other aspects, the present disclosure provides use of an anti-C1q antibody in the manufacture of a medicament for treating or preventing Guillain-Barré Syndrome (GBS) in an individual, wherein the anti-C1q antibody is: i) an isolated anti-C1q antibody comprising a light chain variable domain and a heavy chain variable domain, wherein the light chain variable domain comprises the HVR-L1, HVR-L2, and HVR-L3 of the monoclonal antibody M1 produced by a hybridoma cell line with ATCC Accession Number PTA-120399 or progeny thereof and/or wherein the heavy chain variable domain comprises the HVR-H1, HVR-H2, and HVR-H3 of the monoclonal antibody M1 produced by a hybridoma cell line with ATCC Accession Number PTA-120399 or progeny thereof or ii) an isolated anti-C1q antibody which binds essentially the same C1q epitope as the antibody M1 produced by the hybridoma cell line with ATCC Accession Number PTA-120399 or anti-C1q binding fragments thereof.

In other aspects, the present disclosure provides a method of treating or preventing Guillain-Barré Syndrome (GBS) in an individual, comprising administering to the individual a therapeutically effective amount of an anti-C1s antibody wherein the antibody is: i) an isolated anti-C1s antibody comprising a light chain variable domain and a heavy chain variable domain, wherein the light chain variable domain comprises the HVR-L1, HVR-L2, and HVR-L3 of the monoclonal antibody 5A1 produced by a hybridoma cell line with ATCC Accession Number PTA-120351, or progeny thereof; ii) an isolated anti-C1s antibody comprising a light chain variable domain and a heavy chain variable domain, wherein the heavy chain variable domain comprises the HVR-H1, HVR-H2, and HVR-H3 of the monoclonal antibody 5A1 produced by a hybridoma cell line with ATCC Accession Number PTA-120351, or progeny thereof; iii) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5A1 produced by a hybridoma cell line with ATCC Accession Number PTA-120351 or progeny thereof; iv) an isolated anti-C1s antibody comprising a light chain variable domain and a heavy chain variable domain, wherein the light chain variable domain comprises the HVR-L1, HVR-L2, and HVR-L3 of the monoclonal antibody 5C12 produced by a hybridoma cell line with ATCC Accession Number PTA-120352, or progeny thereof; v) an isolated anti-C1s antibody comprising a light chain variable domain and a heavy chain variable domain, wherein the heavy chain variable domain comprises the HVR-H1, HVR-H2, and HVR-H3 of the monoclonal antibody 5C12 produced by a hybridoma cell line with ATCC Accession Number PTA-120352, or progeny thereof; vi) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5C12 produced by a hybridoma cell line with ATCC Accession Number PTA-120352, or progeny thereof; vii) an isolated murine anti-human C1s monoclonal antibody 5A1 produced by a hybridoma cell line with ATCC Accession Number PTA-120351, or progeny thereof; viii) an isolated murine anti-human C1s monoclonal antibody 5C12 produced by a hybridoma cell line with ATCC Accession Number PTA-120352 or progeny thereof; ix) an isolated anti-C1s antibody which binds essentially the same C1s epitope as the antibody 5A1 produced by a hybridoma cell line with ATCC Accession Number PTA-120351; or x) an isolated anti-C1s antibody which binds essentially the same C1s epitope as the 5C12 antibody produced by a hybridoma cell line with ATCC Accession Number PTA-120352. In other aspects, the present disclosure provides an anti-Cis antibody for use in treating or preventing Guillain-Barré Syndrome (GBS) in an individual, wherein the anti-C1s antibody is: i) an isolated anti-C1s antibody comprising a light chain variable domain and a heavy chain variable domain, wherein the light chain variable domain comprises the HVR-L1, HVR-L2, and HVR-L3 of the monoclonal antibody 5A1 produced by a hybridoma cell line with ATCC Accession Number PTA-120351, or progeny thereof; ii) an isolated anti-C1s antibody comprising a light chain variable domain and a heavy chain variable domain, wherein the heavy chain variable domain comprises the HVR-H1, HVR-H2, and HVR-H3 of the monoclonal antibody 5A1 produced by a hybridoma cell line with ATCC Accession Number PTA-120351, or progeny thereof; iii) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5A1 produced by a hybridoma cell line with ATCC Accession Number PTA-120351 or progeny thereof; iv) an isolated anti-C1s antibody comprising a light chain variable domain and a heavy chain variable domain, wherein the light chain variable domain comprises the HVR-L1, HVR-L2, and HVR-L3 of the monoclonal antibody 5C12 produced by a hybridoma cell line with ATCC Accession Number PTA-120352, or progeny thereof; v) an isolated anti-C1s antibody comprising a light chain variable domain and a heavy chain variable domain, wherein the heavy chain variable domain comprises the HVR-H1, HVR-H2, and HVR-H3 of the monoclonal antibody 5C12 produced by a hybridoma cell line with ATCC Accession Number PTA-120352, or progeny thereof; vi) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5C12 produced by a hybridoma cell line with ATCC Accession Number PTA-120352, or progeny thereof; vii) an isolated murine anti-human C1s monoclonal antibody 5A1 produced by a hybridoma cell line with ATCC Accession Number PTA-120351, or progeny thereof; viii) an isolated murine anti-human C1s monoclonal antibody 5C12 produced by a hybridoma cell line with ATCC Accession Number PTA-120352 or progeny thereof; ix) an isolated anti-C1s antibody which binds essentially the same C1s epitope as the antibody 5A1 produced by a hybridoma cell line with ATCC Accession Number PTA-120351; or x) an isolated anti-C1s antibody which binds essentially the same C1s epitope as the 5C12 antibody produced by a hybridoma cell line with ATCC Accession Number PTA-120352. In other aspects, the present disclosure provides use of an anti-C1s antibody in the manufacture of a medicament for treating or preventing Guillain-Barré Syndrome (GBS) in an individual, wherein the anti-C1s antibody is: i) an isolated anti-C1s antibody comprising a light chain variable domain and a heavy chain variable domain, wherein the light chain variable domain comprises the HVR-L1, HVR-L2, and HVR-L3 of the monoclonal antibody 5A1 produced by a hybridoma cell line with ATCC Accession Number PTA-120351, or progeny thereof; ii) an isolated anti-C1s antibody comprising a light chain variable domain and a heavy chain variable domain, wherein the heavy chain variable domain comprises the HVR-H1, HVR-H2, and HVR-H3 of the monoclonal antibody 5A1 produced by a hybridoma cell line with ATCC Accession Number PTA-120351, or progeny thereof; iii) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5A1 produced by a hybridoma cell line with ATCC Accession Number PTA-120351 or progeny thereof; iv) an isolated anti-C1s antibody comprising a light chain variable domain and a heavy chain variable domain, wherein the light chain variable domain comprises the HVR-L1, HVR-L2, and HVR-L3 of the monoclonal antibody 5C12 produced by a hybridoma cell line with ATCC Accession Number PTA-120352, or progeny thereof; v) an isolated anti-C1s antibody comprising a light chain variable domain and a heavy chain variable domain, wherein the heavy chain variable domain comprises the HVR-H1, HVR-H2, and HVR-H3 of the monoclonal antibody 5C12 produced by a hybridoma cell line with ATCC Accession Number PTA-120352, or progeny thereof; vi) an isolated anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5C12 produced by a hybridoma cell line with ATCC Accession Number PTA-120352, or progeny thereof; vii) an isolated murine anti-human C1s monoclonal antibody 5A1 produced by a hybridoma cell line with ATCC Accession Number PTA-120351, or progeny thereof; viii) an isolated murine anti-human C1s monoclonal antibody 5C12 produced by a hybridoma cell line with ATCC Accession Number PTA-120352 or progeny thereof; ix) an isolated anti-C1s antibody which binds essentially the same C1s epitope as the antibody 5A1 produced by a hybridoma cell line with ATCC Accession Number PTA-120351; or x) an isolated anti-C1s antibody which binds essentially the same C1s epitope as the 5C12 antibody produced by a hybridoma cell line with ATCC Accession Number PTA-120352. In some embodiments, an anti-C1s antibody of this disclosure specifically binds to and neutralizes a biological activity of C1s or the C1s proenzyme. In certain embodiments, the biological activity is C1s binding to C1q, C1s binding to C1r, or C1s binding to C2 or C4. In certain embodiments, the biological activity is the proteolytic enzyme activity of C1s, the conversion of the C1s proenzyme to an active protease, or proteolytic cleavage of C4. In certain embodiments, the biological activity is activation of the classical complement activation pathway, activation of antibody and complement dependent cytotoxicity, or C1F hemolysis.

In certain embodiments that may be combined with any of the preceding embodiments, the individual has GBS. In certain embodiments that may be combined with any of the preceding embodiments, the individual is a human. In certain embodiments that may be combined with any of the preceding embodiments, the antibody binds C1q, C1r, or C1s. In certain embodiments that may be combined with any of the preceding embodiments, the antibody is an anti-C1q antibody. In certain embodiments that may be combined with any of the preceding embodiments, the antibody is an isolated anti-C1q antibody, which binds to a C1q protein and binds to one or more amino acids of the C1q protein within amino acid residues selected from the group consisting of: i) amino acid residues 196-226 of SEQ ID NO:1, or amino acid residues of a C1q protein chain A (C1qA) corresponding to amino acid residues 196-226 (GLFQVVSGGMVLQLQQGDQVWVEKDPKKGHI) of SEQ ID NO:1; ii) amino acid residues 196-221 of SEQ ID NO:1, or amino acid residues of a C1qA corresponding to amino acid residues 196-221 (GLFQVVSGGMVLQLQQGDQVWVEKDP) of SEQ ID. NO:1; iii) amino acid residues 202-221 of SEQ ID NO:1, or amino acid residues of a C1qA corresponding to amino acid residues 202-221 (SGGMVLQLQQGDQVWVEKD) of SEQ ID NO:1; iv) amino acid residues 202-219 of SEQ ID NO:1, or amino acid residues of a C1qA corresponding to amino acid residues 202-219 (SGGMVLQLQQGDQVWVEK) of SEQ ID NO:1; and v) amino acid residues Lys 219 and/or Ser 202 of SEQ ID NO:1, or amino acid residues of a C1qA corresponding Lys 219 and/or Ser 202 of SEQ ID NO:1. In certain embodiments that may be combined with any of the preceding embodiments, the anti-C1q antibody further binds to one or more amino acids of the C1q protein within amino acid residues selected from the group consisting of: i) amino acid residues 218-240 of SEQ ID NO:3 or amino acid residues of a C1q protein chain C (C1qC) corresponding to amino acid residues 218-240 (WLAVNDYYDMVGI QGSDSVFSGF) of SEQ ID NO:3; amino acid residues 225-240 of SEQ ID NO:3 or amino acid residues of a C1qC corresponding to amino acid residues 225-240 (YDMVGI QGSDSVFSGF) of SEQ ID NO:3; iii) amino acid residues 225-232 of SEQ ID NO:3 or amino acid residues of a C1qC corresponding to amino acid residues 225-232 (YDMVGIQG) of SEQ ID NO:3; iv) amino acid residue Tyr 225 of SEQ ID NO:3 or an amino acid residue of a C1qC corresponding to amino acid residue Tyr 225 of SEQ ID NO:3; v) amino acid residues 174-196 of SEQ ID NO:3 or amino acid residues of a C1qC corresponding to amino acid residues 174-196 (HTANLCVLLYRSGVKVVTFCGHT) of SEQ ID NO:3; vi) amino acid residues 184-192 of SEQ ID NO:3 or amino acid residues of a C1qC corresponding to amino acid residues 184-192 (RSGVKVVTF) of SEQ ID NO:3; vii) amino acid residues 185-187 of SEQ ID NO:3 or amino acid residues of a C1qC corresponding to amino acid residues 185-187 (SGV) of SEQ ID NO:3; and viii) amino acid residue Ser 185 of SEQ ID NO:3 or an amino acid residue of a C1qC corresponding to amino acid residue Ser 185 of SEQ ID NO:3.

In certain embodiments that may be combined with any of the preceding embodiments, the antibody is an isolated anti-C1q antibody, which binds to a C1q protein and binds to one or more amino acids of the C1q protein chain A (C1qA) within amino acid residues selected from the group consisting of: i) amino acid residues 196-226 of SEQ ID NO:1, or amino acid residues of a C1q protein chain A (C1qA) corresponding to amino acid residues 196-226 (GLFQVVSGGMVLQLQQGDQVWVEKDPKKGHI) of SEQ ID NO:1; ii) amino acid residues 196-221 of SEQ ID NO:1, or amino acid residues of a C1qA corresponding to amino acid residues 196-221 (GLFQVVSGGMVLQLQQGDQVWVEKDP) of SEQ ID. NO:1; iii) amino acid residues 202-221 of SEQ ID NO:1, or amino acid residues of a C1qA corresponding to amino acid residues 202-221 (SGGMVLQLQQGDQVWVEKD) of SEQ ID NO:1; iv) amino acid residues 202-219 of SEQ ID NO:1, or amino acid residues of a C1qA corresponding to amino acid residues 202-219 (SGGMVLQLQQGDQVWVEK) of SEQ ID NO:1; and v) amino acid residue Lys 219 of SEQ ID NO:1, or an amino acid residue of a C1qA corresponding Lys 219 of SEQ ID NO:1; and wherein the isolated anti-C1q antibody binds to one or more amino acids of the C1q protein chain C (C1qC) within amino acid residues selected from the group consisting of: i) amino acid residues 174-196 of SEQ ID NO:3 or amino acid residues of a C1qC corresponding to amino acid residues 174-196 (HTANLCVLLYRSGVKVVTFCGHT) of SEQ ID NO:3; ii) amino acid residues 184-192 of SEQ ID NO:3 or amino acid residues of a C1qC corresponding to amino acid residues 184-192 (RSGVKVVTF) of SEQ ID NO:3; iii) amino acid residues 185-187 of SEQ ID NO:3 or amino acid residues of a C1qC corresponding to amino acid residues 185-187 (SGV) of SEQ ID NO:3; and iv) amino acid residue Ser 185 of SEQ ID NO:3 or an amino acid residue of a C1qC corresponding to amino acid residue Ser 185 of SEQ ID NO:3. In certain embodiments that may be combined with any of the preceding embodiments, the antibody is an anti-C1q antibody, wherein the anti-C1q antibody binds specifically to both human C1q and mouse C1q. In certain embodiments that may be combined with any of the preceding embodiments, the antibody is an anti-C1q antibody, wherein the anti-C1q antibody has dissociation constant (K_{D}) for human C1q and mouse C1q of less than 100 pM. In certain embodiments that may be combined with any of the preceding embodiments, the antibody is an anti-Cq1q antibody, wherein the anti-C1q antibody specifically binds to and neutralizes a biological activity of C1q. In certain embodiments that may be combined with any of the preceding embodiments, the antibody is an anti-C1q antibody, wherein the biological activity is (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to beta-amyloid, or (10) C1q binding to calreticulin. In certain embodiments that may be combined with any of the preceding embodiments, the antibody is an anti-C1q antibody, wherein the biological activity is (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) Arthus reaction, (11) phagocytosis of synapses or nerve endings, or (12) activation of complement receptor 3 (CR3/C3) expressing cells. In certain embodiments that may be combined with any of the preceding embodiments, the antibody is an anti-C1s antibody. In certain embodiments that may be combined with any of the preceding embodiments, the antibody is an anti-C1s antibody, wherein the anti-C1s antibody specifically binds to and neutralizes a biological activity of C1s or the C1s proenzyme. In certain embodiments that may be combined with any of the preceding embodiments, the antibody is an anti-C1s antibody, wherein said biological activity is (1) C1s binding to C1q, (2) C1s binding to C1r, or (3) C1s binding to C2 or C4. In certain embodiments that may be combined with any of the preceding embodiments, the antibody is an anti-C1s antibody, wherein said biological activity is (1) the proteolytic enzyme activity of C1s, (2) the conversion of the C1s proenzyme to an active protease, or (3) cleavage of C4. In certain embodiments that may be combined with any of the preceding embodiments, the antibody is an anti-C1s antibody, wherein said biological activity is (1) activation of the classical complement activation pathway, (2) activation of antibody and complement dependent cytotoxicity, or (3) C1F hemolysis. In certain embodiments that may be combined with any of the preceding embodiments, the antibody is an anti-C1s antibody, wherein the anti-C1s antibody is a murine antibody. In certain embodiments that may be combined with any of the preceding embodiments, the antibody is an anti-C1s antibody, wherein said antibody is capable of neutralizing at least 30%, at least 50%, or at least 70% of C1F hemolysis. In certain embodiments that may be combined with any of the preceding embodiments, the antibody is an anti-C1r antibody. In certain embodiments that may be combined with any of the preceding embodiments, the antibody is an anti-C1 complex antibody. In certain embodiments that may be combined with any of the preceding embodiments, the antibody binds human C1q, C1r, or C1s. In certain embodiments that may be combined with any of the preceding embodiments, the antibody binds human C1 complex. In certain embodiments that may be combined with any of the preceding embodiments, the antibody is a monoclonal antibody. In certain embodiments that may be combined with any of the preceding embodiments, the antibody is a mouse antibody, a human antibody, a humanized antibody, or a chimeric antibody. In certain embodiments that may be combined with any of the preceding embodiments, the antibody is of the IgG class, including IgG₁, IgG₂, IgG₃, or IgG₄ isotypes. In certain embodiments that may be combined with any of the preceding embodiments, the antibody is an antibody fragment selected from the group consisting of Fab, Fab′-SH, Fv, scFv, and (Fab′)₂ fragments. In certain embodiments that may be combined with any of the preceding embodiments, the antibody is a bispecific antibody recognizing a first antigen and a second antigen. In certain embodiments that may be combined with any of the preceding embodiments, the first antigen is selected from the group consisting of C1q, C1r, C1s, and the C1 complex and the second antigen is an antigen facilitating transport across the blood-brain-barrier. In certain embodiments that may be combined with any of the preceding embodiments, the second antigen is selected from the group consisting of transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005. In certain embodiments that may be combined with any of the preceding embodiments, the antibody inhibits C3c deposition. In certain embodiments that may be combined with any of the preceding embodiments, the antibody inhibits membrane attack complex (MAC) deposition. In certain embodiments that may be combined with any of the preceding embodiments, the antibody inhibits axonal damage formation. In certain embodiments that may be combined with any of the preceding embodiments, the antibody inhibits respiratory muscle damage. In certain embodiments that may be combined with any of the preceding embodiments, the antibody inhibits the classical complement activation pathway by an amount that ranges from at least 30% to at least 99.9%. In certain embodiments that may be combined with any of the preceding embodiments, the antibody inhibits the alternative complement activation pathway by an amount that ranges from at least 30% to at least 99.9%. In certain embodiments that may be combined with any of the preceding embodiments, the antibody inhibits complement-dependent cell-mediated cytotoxicity (CDCC) activation pathway by an amount that ranges from at least 30% to at least 99.9%. In certain embodiments that may be combined with any of the preceding embodiments, the antibody does not inhibit the lectin complement activation pathway. In certain embodiments that may be combined with any of the preceding embodiments, the antibody comprises a dissociation constant (K_{D}) for its corresponding antigen that ranges from 100 nM to 0.005 nM or less than 0.005 nM. In certain embodiments that may be combined with any of the preceding embodiments, the antibody inhibits autoantibody-dependent and complement-dependent cytotoxicity (CDC). In certain embodiments that may be combined with any of the preceding embodiments, the antibody prevents amplification of the alternative complement activation pathway initiated by C1q binding. In certain embodiments that may be combined with any of the preceding embodiments, the antibody comprises an EC₅₀ that ranges from 3 μg/ml to 0.05 μg/ml, or less than 0.05 μg/ml. In certain embodiments that may be combined with any of the preceding embodiments, the antibody does not inhibit autoantibody-dependent cellular cytotoxicity (ADCC). In certain embodiments that may be combined with any of the preceding embodiments, the method further comprises administering to the individual a therapeutically effective amount of a second antibody, wherein the second antibody is selected from the group consisting of the anti-C1q antibody, the anti-C1r antibody, the anti-C1s antibody and the anti-C1 complex antibody. In certain embodiments that may be combined with any of the preceding embodiments, the method further comprises administering to the individual a therapeutically effective amount of an inhibitor of antibody-dependent cellular cytotoxicity (ADCC). In certain embodiments that may be combined with any of the preceding embodiments, the method further comprises administering to the individual a therapeutically effective amount of an inhibitor of the alternative complement activation pathway. In certain embodiments that may be combined with any of the preceding embodiments, the method further comprises administering to the individual an inhibitor of the interaction between the autoantibody and an autoantigen. In certain embodiments that may be combined with any of the preceding embodiments, the antibody binds its corresponding antigen with a binding stoichiometry that ranges from 20:1 to 1.0:1 or less than 1.0:1.

In other aspects, the present disclosure provides a diagnostic kit comprising an antibody of any of the preceding embodiments for treating or preventing Guillain-Barré Syndrome (GBS) in an individual.

It is to be understood that one, some, or all of the properties of the various embodiments described herein may be combined to form other embodiments of the compositions and methods provided herein. These and other aspects of the compositions and methods provided herein will become apparent to one of skill in the art.

### DESCRIPTION OF THE FIGURES

FIG. 1 illustrates the results of an ELISA screen for antibodies specifically binding human C1s or human C1s proenzyme. The binding assays were conducted either in the absence of an anti-C1s antibody (“media”) or in the presence of one of six anti-C1s antibodies (1B4, 3F8, 3G3, 5A1, 5C12, or 7C4). Left columns show binding signals for anti-C1s antibody binding to the C1s protein; middle columns show binding signals for anti-C1s antibody binding to the C1s proenzyme; right columns show binding signals for anti-C1s antibody binding to the human transferrin (HT) negative control protein.

FIG. 2 illustrates the C1s neutralizing activities of anti-C1s antibodies in a C1F hemolytic assay. FIG. 2A illustrates the results of assays conducted with six anti-C1s antibodies (1B4, 3F8, 3G3, 5A1, 5C12, or 7C4) in a single-dose format. FIG. 2B shows the results of C1F hemolytic assays conducted with two anti-C1s antibodies (5A1 and 5C12) in a dose-response format.

FIG. 3 illustrates the C1s neutralizing activities of anti-C1s antibodies in a C4 cleavage assay. The upper panel illustrates the activity of eight anti-C1s antibodies (M241, 3F8, 3A1, 3A2, 2A1, 5C12, 6HK, and 8HK) regarding the inhibition of C4 cleavage at a single concentration. The lower panel illustrates the neutralizing activities of two anti-C1s antibodies (5A1 and 5C12) in a dose-response format.

FIG. 4 illustrates mass spectrometry characterization of C1q antibody complexes. FIG. 4A depicts a mixture of ANN-001 (4A4B11) and C1q showing that ANN-001 monomer at the predicted mass of ˜150 kDa, C1q monomer at the expected mass of ˜460 kDa, and the C1q/ANN-001 1:1 complex at the predicted mass of ˜600 kDa. FIG. 4B depicts a mixture of ANN-005 (M1) and C1q showing that ANN-005 monomer at the predicted mass of ˜150 kDa, C1q monomer at the expected mass of ˜460 kDa, and the C1q/ANN-005 1:1 complex at the predicted mass of ˜600 kDa.

FIG. 5A shows a general schematic representation of the complement cascade, including the three complement activation pathways and the terminal pathway. FIG. 5B shows a schematic of the C1 complex. The C1s and C1r dimers are seen in a complex with the C1q hexamer.

FIG. 6 illustrates how anti-C1 antibodies can be used to prevent GBS-anti-ganglioside antibody dependent complement deposition on diaphragm motor nerve terminals ex vivo. Whole-mount muscles maintained alive in oxygenated Ringer's solution were incubated with the anti-ganglioside monoclonal antibody CGM3 (50 μg/ml) for 2-2.5 h at 32° C., then for 30 min at 4° C. and then equilibrated for 10 min at room temperature, rinsed in Ringer's medium and subsequently exposed to 40% Normal Human Serum (NHS) as a source of complement in Ringer's medium for 1 h at room temperature. Anti-C1 antibodies (100 μg/ml) or the control mAb (100 μg/ml) were mixed with NHS 10 min prior to the incubation of the muscle preparation. C3c was detected by incubation with FITC-labelled rabbit anti-C3c (1/300; Dako, Ely, UK) for 1 h at 4° C., and nerve terminals detected by incubation with TRITC labelled α-bungarotoxin (BTx; 1/750; Sigma, UK) that binds nicotinic acetylcholine receptors (nAChR) on the postsynaptic membrane of the neuromuscular junction (NMJ). Digital images were captured using both a Zeiss Pascal confocal laser scanning microscope and a Zeiss Axio Imager Z1 with ApoTome. Image-analysis measurements were made using ImageJ (NIH) image analysis software. For quantitative analysis of C3c three staining runs were performed on tissue from at least three individual muscle preparations, and quantified as previously described (O'Hanlon et al., 2001) and displayed as box and whisker graphs.

FIG. 7 illustrates that anti-C1q antibodies can suppress complement deposition and preserve axonal integrity in an ex-vivo GBS assay. FIG. 7A shows a quantitative representation of the immunofluorescent labeling of C3c deposition on the explanted diaphragm plotted as a box and whisker plot to represent the spread of the nonparametric data. FIG. 7B depicts images of sections quantitated in FIG. 3A showing TRITC-labelled BTx staining of the nAChR in the top panel, FITC-labelled rabbit anti-C3c labeling in the middle panel, and the merged images in the bottom panel. The tissues are either untreated (no complement added) or treated with a control IgG1 antibody or the anti-C1q antibodies 4A4B11 and M1. FIG. 7C shows a quantitative representation of the amount of axonal neurofilament labeled by the rabbit polyclonal serum 1211. FIG. 7D depicts representative images of NMJ showing the post-synaptic membrane (nAChR) and axon (Nfil) staining in the presence of the anti-C1q and control antibodies.

FIG. 8 shows that the anti-C1q antibody M1 can prevent complement deposition and axonal degradation in an in vivo mouse model of GBS. Balb/c mice (3-4 weeks old, 10-15 g) were injected intraperitoneally with 1.5 mg CGM3, followed 16 h later by an intravascular (i.v.) injection of 200 μg of anti-C1q M1 antibody and intraperitoneal injection of 0.5 ml 100% NHS. After 4 hours the mice were euthanized and diaphragm muscle tissue was dissected and processed for immunohistological analyses. FIG. 8A shows a box and whisker plot of the quantitation of the C3c immunofluorescence at the motor nerve endplate and corresponding images of C3c (green) deposition at the NMJ with post-synaptic membrane on the muscle fluorescently labeled by (BTx red) from each treatment group. FIG. 8B shows a box and whisker plot of the quantitation of the MAC immunofluorescence at the motor nerve endplate and below are images of MAC (green) deposition at the NMJ with muscle fluorescently labeled by BTx (red). FIG. 8C shows quantitation of the neurofilament staining at the NMJ and below corresponding images of the neurofilament staining.

FIG. 9A shows baseline tidal volume readings recorded the day before treatment with anti-C1q antibody M1 or isotype control antibody ACP1 and subsequent readings were taken at 4 h and 6 h post-injury induction. FIG. 9B shows the percentage change from baseline tidal volume for the experimental groups treated with anti-C1q antibody M1 or isotype control antibody ACP1.

### DETAILED DESCRIPTION OF THE INVENTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### DEFINITIONS

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disease, disorder, or condition. A therapeutically effective amount herein may vary according to factors such as the disease state, age, sex, and weight of the patient, and the ability of the anti-C1q, anti-C1s, anti-C1r, and/or anti-C1 complex antibody to elicit a desired response in the individual. A therapeutically effective amount is also one in which any toxic or detrimental effects of the anti-C1q, anti-C1s, anti-C1r, and/or anti-C1 complex antibody are outweighed by the therapeutically beneficial effects.

“Chronic” administration refers to administration of the medicament(s) in a continuous as opposed to acute mode, so as to maintain the initial therapeutic effect (activity) for an extended period of time. “Intermittent” administration refers to treatment that is not consecutively done without interruption, but rather is cyclic in nature.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

As used herein, “autoantibody” means any antibody that recognizes a host antigen, such as AQP4, in an individual having GBS and activates the classical pathway of complement activation. In the first step of this activation process complement factor C1q binds to the autoantibody-autoantigen-immune complex. Autoantibodies may include naturally occurring antibodies, such as serum antibodies from GBS patients (commonly referred to as GBS-IgG) or monoclonal antibodies, such as rAb-53.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and

Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an anti-C1q, anti-C1s, anti-C1r, and/or anti-C1 complex antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

The “variable region” or “variable domain” of an antibody, such as an anti-C1q, anti-C1s, anti-C1r, and/or anti-C1 complex antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-C1q, anti-C1s, anti-C1r, and/or anti-C1 complex antibodies of the present disclosure. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as an anti-C1q, anti-C1s, anti-C1r, and/or anti-C1 complex antibody of the present disclosure, obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-C1q, anti-C1s, anti-C1r, and/or anti-C1 complex antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-C1q, anti-C1s, anti-C1r, and/or anti-C1 complex antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

“Functional fragments” of antibodies, such as anti-C1q, anti-C1s, anti-C1r, and/or anti-C1 complex antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody (immunoglobulin), such as an anti-C1s, anti-C1q, anti-C1r, and/or anti-C1 complex antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies, such as anti-C1q, anti-C1s, anti-C1r, and/or anti-C1 complex antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-C1q, anti-C1s, anti-C1r, and/or anti-C1 complex antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain, such as that of an anti-C1q, anti-C1s, anti-C1r, and/or anti-C1 complex antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position, e.g., of an anti-C1q, anti-C1s, anti-C1r, and/or anti-C1 complex antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-C1q, anti-C1s, anti-C1r, and/or anti-C1 complex antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody, such as an anti-C1q, anti-C1s, anti-C1r, and/or anti-C1 complex antibody of the present disclosure, that is determinative of the presence of the target in the presence of a heterogeneous population of molecules including biological molecules. For example, an antibody, such as an anti-C1q, anti-C1s, anti-C1r, and/or anti-C1 complex antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴M⁻¹, sometimes about 10⁵M⁻¹ or 10⁶M⁻¹, in other instances about 10⁶ M⁻¹ or 10⁷ M⁻¹, about 10⁸M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a complement protein, such as complement factors C1q, C1s, and C1r and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody, such as an anti-C1q, anti-C1s, anti-C1r, and/or anti-C1 complex antibody of the present disclosure that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” molecule or cell is a molecule or a cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature. Isolated molecules therefore are distinguished from molecules existing naturally in cells; isolated cells are distinguished from cells existing naturally in tissues, organs, or individuals. In some embodiments, the isolated molecule is an anti-C1q, anti-C1s, anti-C1r, and/or anti-C1 complex antibody of the present disclosure. In other embodiments, the isolated cell is a host cell or hybridoma cell producing an anti-C1q, anti-C1s, anti-C1r, and/or anti-C1 complex antibody of the present disclosure.

An “isolated” nucleic acid molecule encoding an antibody, such as an anti-C1q, anti-C1s, anti-C1r, and/or anti-C1 complex antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the invention described herein include “comprising,” “consisting,” and “consisting essentially of” aspects and embodiments.

### Overview

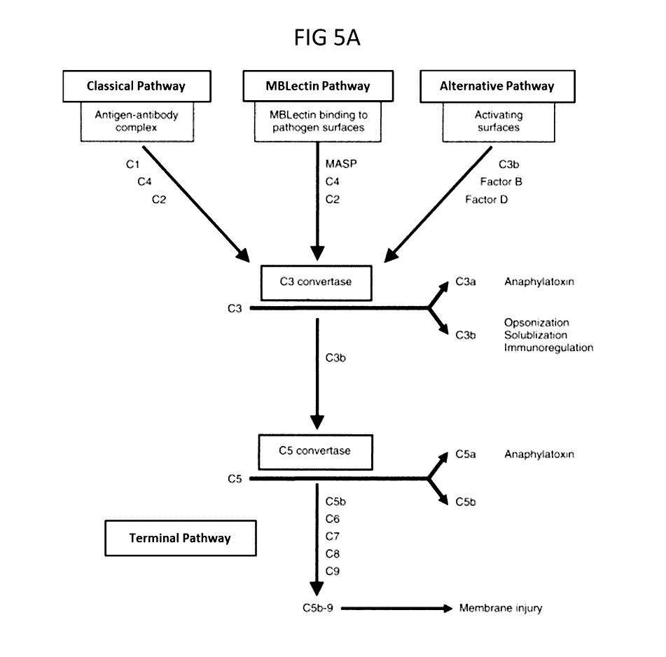

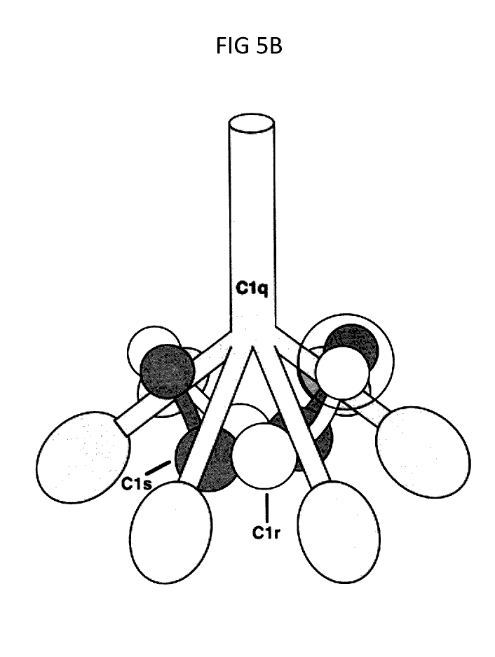

In certain aspects, the present disclosure provides methods of treating, preventing, or reducing risk of Guillain-Barre Syndrome (GBS). Without wishing to be bound by theory, it is believed that inhibition of the classical pathway of complement activation is an effective therapeutic strategy for the treatment of GBS (FIG. 5A). It is further believed that effective strategies for inhibiting the classical pathway include inhibiting the interaction between C1q and autoantibodies (e.g., anti-ganglioside autoantibodies), i inhibiting the interaction between C1q and C1r or C1s, blocking the catalytic activity of C1r or C1s, and blocking the interactions between C1r or C1s and their respective substrates (FIG. 5A). It is also believed that effective agents for the inhibition of the classical complement pathway include neutralizing antibodies for C1q, C1s, C1r, and/or C1 complex (FIG. 5B).

Accordingly, certain aspects of the present disclosure relates to anti-C1q antibodies, anti-C1s antibodies, anti-C1r antibodies, and/or anti-C1 complex antibodies for use in treating, preventing, or reducing risk of Guillain-Barre Syndrome (GBS) in individuals in need thereof.

In one aspect, the present disclosure provides methods for treating or preventing GBS an individual by administering to the individual a therapeutically effective amount of at least one antibody, wherein the at least one antibody is an anti-C1q antibody. In some embodiments, the anti-C1q antibody is a C1q neutralizing antibody. In some embodiments, the anti-C1q antibody binds to C1 complex. In some embodiments, the anti-C1q antibody inhibits the interaction between C1q and an autoantibody, between C1q and C1r, and/or between C1q and C1s. In some embodiments, the individual has GBS. In certain preferred embodiments, the individual is a human.

In another aspect, the present disclosure provides methods for treating or preventing GBS an individual by administering to the individual a therapeutically effective amount of at least one antibody, wherein the at least one antibody is an anti-C1s antibody. In some embodiments, the anti-C1s antibody is a C1s neutralizing antibody. In some embodiments, the anti-C1s antibody binds to C1 complex. In some embodiments, the anti-C1s antibody inhibits the interaction between C1s and C1q, between C1s and C1r, and/or between C1s and its substrates C2 and C4. In some embodiments, the anti-C1s antibody inhibits the catalytic activity of C1s or the processing of pro-C1s into an active protease.

In another aspect, the present disclosure provides methods for treating or preventing GBS an individual by administering to the individual a therapeutically effective amount of at least one antibody, wherein the at least one antibody is an anti-C1r antibody. In some embodiments, the anti-C1r antibody is a C1r neutralizing antibody. In some embodiments, the anti-C1r antibody binds to C1 complex. In some embodiments, the anti-C1r antibody inhibits the interaction between C1r and C1q and/or between C1r and C1s. In some embodiments, the anti-C1r antibody inhibits the catalytic activity of C1r or the processing of pro-C1r to an active protease.

In another aspect, the present disclosure provides methods for treating or preventing GBS an individual by administering to the individual a therapeutically effective amount of at least one antibody, wherein the at least one antibody is an anti-C1 complex antibody. In some embodiments, the anti-C1 complex antibody is a C1 complex neutralizing antibody. In some embodiments, the anti-C1 complex antibody binds to C1q, C1s, and/or C1r. In some embodiments, the anti-C1 complex antibody inhibits C1r activation and/or C1s activation. In some embodiments, the anti-C1 complex antibody prevents the ability of C1r to act on C2 or C4 and/or the ability of C1s to act on C2 or C4. In some embodiments, the anti-C1 complex antibody binds to a combinatorial epitope within the C1 complex, wherein said combinatorial epitope is comprised of C1q and C1s; C1q and C1r; C1r and C1s; or C1q, C1r, and C1s.

Further aspects of the present disclosure provide neutralizing anti-C1s antibodies and uses therefore. In some embodiments, the present disclosure provides neutralizing monoclonal murine anti-C1s antibodies 5A1 and 5C12, which are produced by hybridoma cell lines deposited with ATCC on May 15, 2013 and having ATCC Accession Numbers PTA-120351 and PTA-120352, and antibodies derived from anti-C1s antibodies 5A1 and 5C12. Uses for neutralizing anti-C1s antibodies include, without limitation, the detection of complement factor C1s. Additional non-limiting uses include the inhibition of the classical pathway of complement activation, e.g., in cases where the classical complement pathway is activated by autoantibodies, such as anti-ganglioside autoantibodies. Further non-limiting uses for neutralizing anti-C1s antibodies include the diagnosis and treatment of disorders associated with increased activation of the classical complement pathway, in particular autoimmune disorders, such as GBS, and neurodegenerative disorders, including neurodegenerative disorders associated with synapse loss.

In a further aspect, the present disclosure provides an anti-C1s monoclonal antibody which binds to and neutralizes a biological activity of C1s. In some embodiments, the anti-C1s antibodies of this disclosure also bind to the C1s proenzyme. The neutralizing anti-C1s antibodies may neutralize, without limitation, one or more biological activities of C1s. Such biological activities include, without limitation, C1s binding to C1q or C1s binding to C1r, as well as C1s binding to C2 or C4. Other non-limiting biological activities of C1s include the proteolytic enzyme activity of C1s or the conversion of the C1s proenzyme (C1s-pro) to an active C1s protease. Other biological activities include the cleavage of C4. Other non-limiting biological activities of C1s include the activation of the classical complement activation pathway, the activation of antibody and complement dependent cytotoxicity, and C1F hemolysis. In some embodiments, the anti-C1s antibodies of this disclosure may bind to C1 complex.

In another aspect, the present disclosure provides an isolated nucleic acid molecule encoding an antibody of this disclosure.

The present disclosure also provides isolated host cells containing a nucleic acid molecule that encodes an antibody of this disclosure. In some embodiments, isolated host cell lines are provided that can produce the neutralizing monoclonal murine antibodies 5A1 and 5C12. Such isolated host cell lines were deposited with ATCC on May 15, 2013 and have ATCC Accession Numbers PTA-120351 and PTA-120352. Additionally, pharmaceutical compositions are provided containing C1s neutralizing antibodies of this disclosure in combination with pharmaceutically acceptable carriers.

The present disclosure further provides methods of using the C1s neutralizing antibodies of this disclosure to treat or prevent an autoimmune or neurodegenerative disease in a subject in need of such treatment, to detect synapses in an individual having an autoimmune or neurodegenerative disease, and to detect synapses in a biological sample. The present disclosure also provides diagnostic kits containing the C1s neutralizing antibodies of this disclosure.

### Complement Proteins

The methods of this disclosure involve administering or using antibodies that specifically recognize complement factors C1q, C1s, C1r, and/or the C1 complex of the classical complement activation pathway. Certain aspects of the present disclosure further involve antibodies that specifically recognize complement factors C1q and/or C1q in the C1 complex, and C1s and/or C1s in the C1 complex of the classical complement activation pathway. The recognized complement factors may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

As used herein “complement factors C1q, C1s, or C1r” refers to both wild type sequences and naturally occurring variant sequences.

C1q

A non-limiting example of a complement factor C1q recognized by antibodies of this invention is human C1q, including the three polypeptide chains A, B, and C:

C1q, chain A (homo sapiens), Accession No. Protein Data Base: NP_057075.1; GenBank No.: NM_015991:

<gi|7705753|ref|NP_057075.1| complement C1q
subcomponent subunit A precursor [Homo sapiens]
(SEQ ID NO: 1)
MEGPRGWLVLCVLAISLASMVTEDLCRAPDGKKGEAGRPGRRGRPGLKGE
QGEPGAPGIRTGIQGLKGDQGEPGPSGNPGKVGYPGPSGPLGARGIPGIK
GTKGSPGNIKDQPRPAFSAIRRNPPMGGNVVIFDTVITNQEEPYQNHSGR
FVCTVPGYYYFTFQVLSQWEICLSIVSSSRGQVRRSLGFCDTTNKGLFQV
VSGGMVLQLQQGDQVWVEKDPKKGHIYQGSEADSVFSGFLIFPSA

C1q, chain B (homo sapiens), Accession No. Protein Data Base: NP_000482.3; GenBank No.: NM_000491.3:

>gi|87298828|ref|NP_000482.3| complement C1q
subcomponent subunit B precursor [Homo sapiens]
(SEQ ID NO: 2)
MMMKIPWGSIPVLMLLLLLGLIDISQAQLSCTGPPAIPGIPGIPGTPGPD
GQPGTPGIKGEKGLPGLAGDHGEFGEKGDPGIPGNPGKVGPKGPMGPKGG
PGAPGAPGPKGESGDYKATQKIAFSATRTINVPLRRDQTIRFDHVITNMN
NNYEPRSGKFTCKVPGLYYFTYHASSRGNLCVNLMRGRERAQKVVTFCDY
AYNTFQVTTGGMVLKLEQGENVFLQATDKNSLLGMEGANSIFSGFLLFPD
MEA

C1q, chain C (homo sapiens), Accession No. Protein Data Base: NP_001107573.1; GenBank No.: NM_001114101.1:

>gi|166235903|ref|NP_001107573.1| complement C1q
subcomponent subunit C precursor [Homo sapiens]
(SEQ ID NO: 3)
MDVGPSSLPHLGLKLLLLLLLLPLRGQANTGCYGIPGMPGLPGAPGKDGY
DGLPGPKGEPGIPAIPGIRGPKGQKGEPGLPGHPGKNGPMGPPGMPGVPG
PMGIPGEPGEEGRYKQKFQSVFTVTRQTHQPPAPNSLIRFNAVLTNPQGD
YDTSTGKFTCKVPGLYYFVYHASHTANLCVLLYRSGVKVVTFCGHTSKTN
QVNSGGVLLRLQVGEEVWLAVNDYYDMVGIQGSDSVFSGFLLFPD

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q.

C1s

A non-limiting example of a complement factor C1s recognized by antibodies of this invention is human C1s (Accession No. Protein Data Base: NP_958850.1; GenBank No.: NM_201442.2):

>gi|41393602|ref|NP_958850.1| complement C1s
subcomponent precursor [Homo sapiens]
(SEQ ID NO: 4)
MWCIVLFSLLAWVYAEPTMYGEILSPNYPQAYPSEVEKSWDIEVPEGYGI
HLYFTHLDIELSENCAYDSVQIISGDTEEGRLCGQRSSNNPHSPIVEEFQ
VPYNKLQVIFKSDFSNEERFTGFAAYYVATDINECTDFVDVPCSHFCNNF
IGGYFCSCPPEYFLHDDMKNCGVNCSGDVFTALIGEIASPNYPKPYPENS
RCEYQIRLEKGFQVVVTLRREDFDVEAADSAGNCLDSLVFVAGDRQFGPY
CGHGFPGPLNIETKSNALDIIFQTDLTGQKKGWKLRYHGDPMPCPKEDTP
NSVWEPAKAKYVFRDVVQITCLDGFEVVEGRVGATSFYSTCQSNGKWSNS
KLKCQPVDCGIPESIENGKVEDPESTLFGSVIRYTCEEPYYYMENGGGGE
YHCAGNGSWVNEVLGPELPKCVPVCGVPREPFEEKQRIIGGSDADIKNFP
WQVFFDNPWAGGALINEYWVLTAAHVVEGNREPTMYVGSTSVQTSRLAKS
KMLTPEHVFIHPGWKLLEVPEGRTNFDNDIALVRLKDPVKMGPTVSPICL
PGTSSDYNLMDGDLGLISGWGRTEKRDRAVRLKAARLPVAPLRKCKEVKV
EKPTADAEAYVFTPNMICAGGEKGMDSCKGDSGGAFAVQDPNDKTKFYAA
GLVSWGPQCGTYGLYTRVKNYVDWIMKTMQENSTPRED

Accordingly, an anti-C1s antibody of the present disclosure may bind to human C1s or a homolog thereof, such as mouse, rat, rabbit, monkey, dog, cat, cow, horse, camel, sheep, goat, or pig C1s.

C1r

A non-limiting example of a complement factor C1r recognized by antibodies of this invention is human C1r (Accession No. Protein Data Base: NP_001724.3; GenBank No.: NM_001733.4):

>gi|66347875|ref|NP_001724.3| complement C1r
subcomponent precursor [Homo sapiens]
(SEQ ID NO: 5)
MWLLYLLVPALFCRAGGSIPIPQKLFGEVTSPLFPKPYPNNFETTTVITV
PTGYRVKLVFQQDLEPSEGCFYDYVKISADKKSLGRFCGQLGSPLGNPPG
KKEFMSQGNKMLLTFHTDFSNEENGTIMFYKGFLAYYQAVDLDECASRSK
LGEEDPQPQCQHLCHNYVGGYFCSCRPGYELQEDRHSCQAECSSELYTEA
SGYISSLEYPRSYPPDLRCNYSIRVERGLTLHLKFLEPFDIDDHQQVHCP
YDQLQIYANGKNIGEFCGKQRPPDLDTSSNAVDLLFFTDESGDSRGWKLR
YTTEIIKCPQPKTLDEFTIIQNLQPQYQFRDYFIATCKQGYQLIEGNQVL
HSFTAVCQDDGTWHRAMPRCKIKDCGQPRNLPNGDFRYTTTMGVNTYKAR
IQYYCHEPYYKMQTRAGSRESEQGVYTCTAQGIWKNEQKGEKIPRCLPVC
GKPVNPVEQRQRIIGGQKAKMGNFPWQVFTNIHGRGGGALLGDRWILTAA
HTLYPKEHEAQSNASLDVFLGHTNVEELMKLGNHPIRRVSVHPDYRQDES
YNFEGDIALLELENSVTLGPNLLPICLPDNDTFYDLGLMGYVSGFGVMEE
KIAHDLRFVRLPVANPQACENWLRGKNRMDVFSQNMFCAGHPSLKQDACQ
GDSGGVFAVRDPNTDRWVATGIVSWGIGCSRGYGFYTKVLNYVDWIKKEM
EEED

Accordingly, an anti-C1r antibody of the present disclosure may bind to human C1r or a homolog thereof, such as mouse, rat, rabbit, monkey, dog, cat, cow, horse, camel, sheep, goat, or pig C1r.

Anti-C1q, anti-C1s, and anti-C1r Antibodies

The antibodies of the present disclosure recognize complement factors C1q, C1r, and/or C1s; and/or the C1 complex of the classical complement activation pathway. In some embodiments, the antibodies neutralize the activity of complement factors C1q, C1r, and/or C1s. In some embodiments, the antibodies inhibit the interaction between complement factors C1q, C1r, and/or C1s and autoantibodies, other complement factors, or complement protease substrates such as C2 and C4. In some embodiments, the antibodies inhibit the catalytic activity of the serine proteases C1s and C1r or inhibit the processing of a serine protease pro-form to an active protease. In some embodiments the antibodies inhibit the classical pathway. In certain embodiments the antibodies further inhibit the alternative pathway. In some embodiments, the antibodies inhibit autoantibody- and complement-dependent cytotoxicity (CDC). In some embodiments, the antibodies inhibit complement-dependent cell-mediated cytotoxicity (CDCC).

Anti-C1q Antibodies

The anti-C1q antibodies of this disclosure recognize and bind to C1q and/or C1q in the C1 complex of the classical complement activation pathway. In some embodiments, the anti-C1q antibodies specifically bind to a human C1q. In some embodiments, the anti-C1q antibodies specifically bind to a human and a mouse C1q. In some embodiments, the anti-C1q antibodies specifically bind to a human, a mouse, and a rat C1q.

In some embodiments, the anti-C1q antibodies of this disclosure neutralize a biological activity of complement factor C1q. In some embodiments, the antibodies inhibit the interaction between complement factor C1q and other complement factors (such as C1r or C1s) or between C1q and an antibody (such as an autoantibody). In some embodiments, the antibodies inhibit the interaction between complement factor C1q and a non-complement factor. A non-complement factor may include phosphatidylserine, pentraxin-3, C-reactive protein (CRP), globular C1q receptor (gC1qR), complement receptor 1 (CR1), β-amyloid, and calreticulin. In some embodiments, the antibodies inhibit the classical complement activation pathway. In certain embodiments, the antibodies further inhibit the alternative pathway. In some embodiments, the antibodies inhibit autoantibody- and complement-dependent cytotoxicity (CDC). In some embodiments, the antibodies inhibit complement-dependent cell-mediated cytotoxicity (CDCC). In some embodiments, the antibodies inhibit B-cell antibody production, dendritic cell maturation, T-cell proliferation, cytokine production, or microglia activation. In some embodiments, the antibodies inhibit the Arthus reaction. In some embodiments, the antibodies inhibit phagocytosis of synapses or nerve endings. In some embodiments, the antibodies inhibit the activation of complement receptor 3 (CR3/C3) expressing cells.

The functional properties of the anti-C1q antibodies of this invention, such as dissociation constants for antigens, inhibition of protein-protein interactions (e.g., C1q-autoantibody interactions), inhibition of autoantibody-dependent and complement-dependent cytotoxicity (CDC), inhibition of complement-dependent cell-mediated cytotoxicity (CDCC), or lesion formation, may, without limitation, be measured in in vitro, ex vivo, or in vivo experiments.

The functional properties of the antibodies of this invention, such as dissociation constants for antigens, inhibition of protein-protein interactions (e.g., C1q-autoantibody interactions), inhibition of autoantibody-dependent and complement-dependent cytotoxicity (CDC), inhibition of complement-dependent cell-mediated cytotoxicity (CDCC), and/or lesion formation, may, without limitation, be measured by in vitro, ex vivo, or in vivo experiments.

The dissociation constants (K_{D}) of the anti-C1q antibodies for C1q may be less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.05 nM, less than 0.01 nM, or less than 0.005 nM. In some embodiments, dissociation constants are less than 100 pM. In certain embodiments, the dissociation constants of the anti-C1q antibody are less than 100 pM for human C1q and less than 100 pM for mouse C1q. Antibody dissociation constants for antigens other than C1q may be least 5-fold, at least 10-fold, at least 100-fold, at least 1,000-fold, at least 10,000-fold, or at least 100,000-fold higher that the dissociation constants for C1q. For example, the dissociation constant of a C1q antibody of this disclosure may be at least 1,000-fold higher for C1s than for C1q. Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. Dissociation constants (K_{D}) of the anti-C1q antibodies for C1q may be determined, e.g., using full-length antibodies or antibody fragments, such as Fab fragments.

The anti-C1q antibodies of this disclosure may bind to C1q antigens derived from any organism having a complement system, including any mammalian organism such as human, mouse, rat, rabbit, monkey, dog, cat, cow, horse, camel, sheep, goat, or pig. In some embodiments, the anti-C1q antibodies bind specifically to epitopes on human C1q. In some embodiments, the anti-C1q antibodies specifically bind to epitopes on both human and mouse C1q. In some embodiments, the anti-C1q antibodies specifically bind to epitopes on human, mouse, and rat C1q.

In some embodiments, the anti-C1q antibody inhibits the interaction between C1q and an autoantibody. In certain embodiments, the autoantibody recognizes gangliosides (e.g., anti-GQ1b). In some embodiments, the anti-C1q antibody inhibits the interaction between C1q and C1r, or between C1q and C1s, or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain. In some embodiments, the anti-C1q antibody binds to the globular domain of the C1q A-, B-, or C-chain. In other embodiments, the anti-C1q antibody binds to the collagen-like domain of the C1q A-, B-, or C-chain.

In some embodiments, provided herein is an anti-C1q antibody that binds to an epitope of C1q that is the same as or overlaps with the C1q epitope bound by another antibody of this disclosure. In certain embodiments, provided herein is an anti-C1q antibody that binds to an epitope of C1q that is the same as or overlaps with the C1q epitope bound by anti-C1q antibody M1. In some embodiments, the anti-C1q antibody competes with another antibody of this disclosure for binding to C1q. In certain embodiments, the anti-C1q antibody competes with anti-C1q antibody M1 or an antigen-binding fragment thereof for binding to C1q. Methods that may be used to determine which C1q epitope of an anti-C1q antibody binds to, or whether two antibodies bind to the same or an overlapping epitope, may include, without limitation, X-ray crystallography, NMR spectroscopy, Alanine-Scanning Mutagenesis, the screening of peptide libraries that include C1q-derived peptides with overlapping C1q sequences, and competition assays.

In some embodiments, provided herein are anti-C1q antibodies that compete with antibody M1 or an anti-C1q antibody described herein for binding to C1q. Competition assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels.

Competitive anti-C1q antibodies encompassed herein are antibodies that inhibit (i.e., prevent or interfere with in comparison to a control) or reduce the binding of any anti-C1q antibody of this disclosure (such as M1 or an antigen-binding fragment of M1) to C1q by at least 50%, 60%, 70%, 80%, 90% and 95% at 1 μM or less. For example, the concentration competing antibody in the competition assay may be at or below the K_{D} of antibody M1 or an antigen-binding fragment of M1. Competition between binding members may be readily assayed in vitro for example using ELISA and/or by monitoring the interaction of the antibodies with C1q in solution. The exact means for conducting the analysis is not critical. C1q may be immobilized to a 96-well plate or may be placed in a homogenous solution. In specific embodiments, the ability of unlabeled candidate antibody(ies) to block the binding of the labeled anti-C1q antibody, e.g. M1, can be measured using radioactive, enzyme or other labels. In the reverse assay, the ability of unlabeled antibodies to interfere with the interaction of a labeled anti-C1q antibody with C1q wherein said labeled anti-C1q antibody, e.g., M1, and C1q are already bound is determined. The readout is through measurement of bound label. C1q and the candidate antibody(ies) may be added in any order or at the same time.

In some embodiments, the anti-C1q antibody inhibits the interaction between C1q and an autoantibody. In some embodiments, the anti-C1q antibody is murine anti-human C1q monoclonal antibody M1, which is produced by a hybridoma cell line deposited with ATCC on Jun. 6, 2013 with ATCC Accession Number PTA-120399.

In some embodiments, the anti-C1q antibody is an isolated antibody which binds essentially the same C1q epitope as M1. In some embodiments, the anti-C1q antibody is an isolated antibody comprising the HVR-L1, HVR-L2, and HVR-L3 of the light chain variable domains of monoclonal antibody M1 produced by the hybridoma cell line deposited with ATCC on Jun. 6, 2013 with ATCC Accession Number PTA-120399, or progeny thereof. In some embodiments, the anti-C1q antibody is an isolated antibody comprising the HVR-H1, HVR-H2, and HVR-H3 of the heavy chain variable domains of monoclonal antibody M1 produced by the hybridoma cell line deposited with ATCC on Jun. 6, 2013 with ATCC Accession Number PTA-120399, or progeny thereof. In some embodiments, the anti-C1q antibody is an isolated antibody comprising the HVR-L1, HVR-L2, and HVR-L3 of the light chain variable domains and the HVR-H1, HVR-H2, and HVR-H3 of the heavy chain variable domains of monoclonal antibody M1 produced by the hybridoma cell line deposited with ATCC on Jun. 6, 2013 with ATCC Accession Number PTA-120399, or progeny thereof.

In some embodiments, the anti-C1q antibody binds to a C1q protein and binds to one or more amino acids of the C1q protein within amino acid residues selected from (a) amino acid residues 196-226 of SEQ ID NO:1, or amino acid residues of a C1q protein chain A (C1 qA) corresponding to amino acid residues 196-226 (GLFQVVSGGMVLQLQQGDQVWVEKDPKKGHI) of SEQ ID NO:1; (b) amino acid residues 196-221 of SEQ ID NO:1, or amino acid residues of a C1qA corresponding to amino acid residues 196-221 (GLFQVVSGGMVLQLQQGDQVWVEKDP) of SEQ ID. NO:1; (c) amino acid residues 202-221 of SEQ ID NO:1, or amino acid residues of a C1qA corresponding to amino acid residues 202-221 (SGGMVLQLQQGDQVWVEKD) of SEQ ID NO:1; (d) amino acid residues 202-219 of SEQ ID NO:1, or amino acid residues of a C1qA corresponding to amino acid residues 202-219 (SGGMVLQLQQGDQVWVEK) of SEQ ID NO:1; and (e) amino acid residues Lys 219 and/or Ser 202 of SEQ ID NO:1, or amino acid residues of a C1qA corresponding Lys 219 and/or Ser 202 of SEQ ID NO:1.

In some embodiments, the antibody further binds to one or more amino acids of the C1q protein within amino acid residues selected from: (a) amino acid residues 218-240 of SEQ ID NO:3 or amino acid residues of a C1q protein chain C (C1qC) corresponding to amino acid residues 218-240 (WLAVNDYYDMVGI QGSDSVFSGF) of SEQ ID NO:3; (b) amino acid residues 225-240 of SEQ ID NO:3 or amino acid residues of a C1qC corresponding to amino acid residues 225-240 (YDMVGI QGSDSVFSGF) of SEQ ID NO:3; (c) amino acid residues 225-232 of SEQ ID NO:3 or amino acid residues of a C1qC corresponding to amino acid residues 225-232 (YDMVGIQG) of SEQ ID NO:3; (d) amino acid residue Tyr 225 of SEQ ID NO:3 or an amino acid residue of a C1qC corresponding to amino acid residue Tyr 225 of SEQ ID NO:3; (e) amino acid residues 174-196 of SEQ ID NO:3 or amino acid residues of a C1qC corresponding to amino acid residues 174-196 (HTANLCVLLYRSGVKVVTFCGHT) of SEQ ID NO:3; (f) amino acid residues 184-192 of SEQ ID NO:3 or amino acid residues of a C1qC corresponding to amino acid residues 184-192 (RSGVKVVTF) of SEQ ID NO:3; (g) amino acid residues 185-187 of SEQ ID NO:3 or amino acid residues of a C1qC corresponding to amino acid residues 185-187 (SGV) of SEQ ID NO:3; (h) amino acid residue Ser 185 of SEQ ID NO:3 or an amino acid residue of a C1qC corresponding to amino acid residue Ser 185 of SEQ ID NO:3.

In certain embodiments, the anti-C1q antibody binds to amino acid residue Lys 219 and Ser 202 of the human C1qA as shown in SEQ ID NO:1 or amino acids of a human C1qA corresponding to Lys 219 and Ser 202 as shown in SEQ ID NO:1, and amino acid residue Tyr 225 of the human C1qC as shown in SEQ ID NO:3 or an amino acid residue of a human C1qC corresponding to Tyr 225 as shown in SEQ ID NO:3. In certain embodiments, the anti-C1q antibody binds to amino acid residue Lys 219 of the human C1qA as shown in SEQ ID NO:1 or an amino acid residue of a human C1qA corresponding to Lys 219 as shown in SEQ ID NO:1, and amino acid residue Ser 185 of the human C1qC as shown in SEQ ID NO:3 or an amino acid residue of a human C1qC corresponding to Ser 185 as shown in SEQ ID NO:3.

In some embodiments, the anti-C1q antibody binds to a C1q protein and binds to one or more amino acids of the C1q protein within amino acid residues selected from: (a) amino acid residues 218-240 of SEQ ID NO:3 or amino acid residues of a C1qC corresponding to amino acid residues 218-240 (WLAVNDYYDMVGI QGSDSVFSGF) of SEQ ID NO:3; (b) amino acid residues 225-240 of SEQ ID NO:3 or amino acid residues of a C1qC corresponding to amino acid residues 225-240 (YDMVGI QGSDSVFSGF) of SEQ ID NO:3; (c) amino acid residues 225-232 of SEQ ID NO:3 or amino acid residues of a C1qC corresponding to amino acid residues 225-232 (YDMVGIQG) of SEQ ID NO:3; (d) amino acid residue Tyr 225 of SEQ ID NO:3 or an amino acid residue of a C1qC corresponding to amino acid residue Tyr 225 of SEQ ID NO:3; (e) amino acid residues 174-196 of SEQ ID NO:3 or amino acid residues of a C1qC corresponding to amino acid residues 174-196 (HTANLCVLLYRSGVKVVTFCGHT) of SEQ ID NO:3; (f) amino acid residues 184-192 of SEQ ID NO:3 or amino acid residues of a C1qC corresponding to amino acid residues 184-192 (RSGVKVVTF) of SEQ ID NO:3; (g) amino acid residues 185-187 of SEQ ID NO:3 or amino acid residues of a C1qC corresponding to amino acid residues 185-187 (SGV) of SEQ ID NO:3; (h) amino acid residue Ser 185 of SEQ ID NO:3 or an amino acid residue of a C1qC corresponding to amino acid residue Ser 185 of SEQ ID NO:3.

In some embodiments, the anti-C1q antibody inhibits the interaction between C1q and an autoantibody at a stoichiometry of less than 2.5:1; 2.0:1; 1.5:1; or 1.0:1. In other embodiments, the anti-C1q antibody binds to C1q with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1q antibody binds C1q with a binding stoichiometry that ranges from 20:1 to 1.0:1 or less than 1.0:1. In certain embodiments, the anti-C1q antibody binds C1q with a binding stoichiometry that ranges from 6:1 to 1.0:1 or less than 1.0:1. In certain embodiments, the anti-C1q antibody binds C1q with a binding stoichiometry that ranges from 2.5:1 to 1.0:1 or less than 1.0:1. In some embodiments, the anti-C1q antibody inhibits the interaction between C1q and C1r, or between C1q and C1s, or between C1q and both C1r and C1s. In some embodiments, the anti-C1q antibody inhibits the interaction between C1q and C1r, between C1q and C1s, and/or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain. In some embodiments, the anti-C1q antibody binds to the C1q A-chain, the C1q B-chain and/or the C1q C-chain. In some embodiments, the anti-C1q antibody binds to the globular domain of the C1q A-chain, B-chain, and/or C-chain. In other embodiments, the anti-C1q antibody binds to the collagen-like domain of the C1q A-chain, the C1q B-chain, and/or the C1q C-chain.

In some embodiments, the anti-C1q antibodies of this disclosure inhibit C1F hemolysis (also referred to as CH50 hemolysis) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the anti-C1q antibodies of this disclosure are absent or wherein control antibodies are used that do not bind to a complement factor or another antibody such as an autoantibody. Methods for measuring C1F hemolysis are well known in the art. The EC₅₀ values for anti-C1q antibodies of this disclosure with respect to C1F hemolysis may be less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml. In some embodiments, the anti-C1q antibodies of this disclosure neutralize at least 50% of C1F hemolysis at a dose of less than 200 ng/ml, less than 100 ng/ml, less than 50 ng/ml, or less than 20 ng/ml. In some embodiments, the anti-C1q antibodies of this disclosure neutralize C1F hemolysis at approximately equimolar concentrations of C1q and the anti-C1q antibody. In some embodiments, the anti-C1q antibodies of this disclosure neutralize hemolysis in a human C1F hemolysis assay. In some embodiments, the anti-C1q antibodies of this disclosure neutralize hemolysis in a human, mouse, and rat C1F hemolysis assay.

In some embodiments, the alternative pathway may amplify CDC initiated by C1q binding and subsequent C1s activation; in at least some of these embodiments, the anti-C1q antibodies of this disclosure inhibit the alternative pathway by at least 30%, at least 40%, at least 50%, at least 60%, at least 70%, at least 80%, at least 90%, at least 95%, or at least 99%, or by an amount that ranges from at least 30% to at least 99%, relative to a control wherein the anti-C1q antibodies of this disclosure are absent.

Anti-C1s Antibodies

The anti-C1s antibodies of this disclosure recognize and bind to complement factor C1s and/or C1s in the C1 complex of the classical complement activation pathway.

In some embodiments, the anti-C1s antibodies neutralize an activity of complement factor C1s. In some embodiments, the anti-C1s antibodies inhibit the interaction between complement factor C1s and other complement factors, such as C1q or C1r, or complement protease substrates, such as C4. In some embodiments, the anti-C1s antibodies inhibit the catalytic activity of the serine protease C1s or inhibit the processing of a serine protease pro-form to an active protease. In some embodiments the anti-C1s antibodies inhibit the classical pathway. In certain embodiments the antibodies further inhibit the alternative pathway. In some embodiments, the anti-C1s antibodies inhibit autoantibody- and complement-dependent cytotoxicity (CDC). In some embodiments, the anti-C1s antibodies inhibit complement-dependent cell-mediated cytotoxicity (CDCC).

The functional properties of the anti-C1s antibodies of this invention, such as dissociation constants for antigens, inhibition of protein-protein interactions (e.g., C1s-C1q interactions), inhibition of autoantibody-dependent and complement-dependent cytotoxicity (CDC), inhibition of complement-dependent cell-mediated cytotoxicity (CDCC), or lesion formation, may, without limitation, be measured in in vitro, ex vivo, or in vivo experiments.

The dissociation constants (K_{D}) of the anti-C1s antibodies for C1s may be less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.05 nM, less than 0.01 nM, or less than 0.005 nM. Preferably, dissociation constants are less than 20 nM. Antibody dissociation constants for antigens other than C1s may be at least 5-fold, at least 10-fold, at least 100-fold, at least 1,000-fold, at least 10,000-fold, or at least 100,000-fold higher that the dissociation constants for their respective antigens. For example, the dissociation constant of a C1s antibody of this disclosure may be at least 1,000-fold higher for C1q than for C1s. Dissociation constants may be determined through any analytical technique, including any biochemical or biophysical technique such as surface plasmon resonance (SPR), isothermal titration calorimetry (ITC), differential scanning calorimetry (DSC), circular dichroism (CD), stopped-flow analysis, and colorimetric or fluorescent protein melting analyses. Dissociation constants (K_{D}) of the anti-C1s antibodies for their respective antigens may be determined, e.g., using full-length antibodies or antibody fragments, such as Fab fragments. The antibodies of this disclosure may bind to C1s antigens derived from any organism having a complement system, including any mammalian organism such as human, mouse, rat, rabbit, monkey, dog, cat, cow, horse, camel, sheep, goat, or pig. In preferred embodiments, the antibodies of this disclosure bind to epitopes comprising amino acid residues on human C1s.

In some embodiments, provided herein are anti-C1s antibodies that compete with antibody 5A1 or 5C12, or an anti-C1s antibody described herein for binding to C1s. Competition assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels.

Competitive anti-C1s antibodies encompassed herein are antibodies that inhibit (i.e., prevent or interfere with in comparison to a control) or reduce 5A1, 5C12, or an anti-C1s antibody described herein binding to C1s by at least 50%, 60%, 70%, and 80% in order of increasing preference (even more preferably, at least 90% and, most preferably, at least 95%) at 1 μM or less with 5A1, 5C12, or an anti-C1s antibody described herein at or below its K_{D}. Competition between binding members may be readily assayed in vitro for example using ELISA and/or by monitoring the interaction of the antibodies with C1s in solution. The exact means for conducting the analysis is not critical. C1s may be immobilized to a 96-well plate or may be placed in a homogenous solution. In specific embodiments, the ability of unlabeled candidate antibody or antibodies to block the binding of labeled 5A1 or 5C12 can be measured using radioactive, enzyme, or other labels. In the reverse assay, the ability of unlabeled antibodies to interfere with the interaction of labeled 5A1 or 5C12 with C1s wherein said 5A1 or 5C12 and C1s are already bound is determined. The readout is through measurement of bound label. C1s and the candidate antibody or antibodies may be added in any order or at the same time.

In some preferred embodiments, the anti-C1s antibody is murine anti-human C1s monoclonal antibody 5A1, which is produced by a hybridoma cell line deposited with ATCC on May 15, 2013 having ATCC Accession Number PTA-120351. In some embodiments, the anti-C1s antibody is an isolated antibody which binds essentially the same C1s epitope as 5A1. In some embodiments, the anti-C1s antibody is an isolated antibody comprising the HVR-L1, HVR-L2, and HVR-L3 of the light chain variable domains of monoclonal antibody 5A1 produced by the hybridoma cell line deposited with ATCC on May 15, 2013 having ATCC Accession Number PTA-120351, or progeny thereof. In some embodiments, the anti-C1s antibody is an isolated antibody comprising the HVR-H1, HVR-H2, and HVR-H3 of the heavy chain variable domains of monoclonal antibody 5A1 produced by the hybridoma cell line deposited with ATCC on May 15, 2013 having ATCC Accession Number PTA-120351, or progeny thereof. In some embodiments, the anti-C1s antibody is an isolated antibody comprising the HVR-L1, HVR-L2, and HVR-L3 of the light chain variable domains and the HVR-H1, HVR-H2, and HVR-H3 of the heavy chain variable domains of monoclonal antibody 5A1 produced by the hybridoma cell line deposited with ATCC on May 15, 2013 having ATCC Accession Number PTA-120351, or progeny thereof.

In some preferred embodiments, the anti-C1s antibody is murine anti-human C1s monoclonal antibody 5C12, which is produced by a hybridoma cell line deposited with ATCC on May 15, 2013 having ATCC Accession Number PTA-120352. In some embodiments, the anti-C1s antibody is an isolated antibody which binds essentially the same C1s epitope as 5C12. In some embodiments, the anti-C1s antibody is an isolated antibody comprising the HVR-L1, HVR-L2, and HVR-L3 of the light chain variable domains of monoclonal antibody 5C12 produced by the hybridoma cell line deposited with ATCC on May 15, 2013 having ATCC Accession Number PTA-120352, or progeny thereof. In some embodiments, the anti-C1s antibody is an isolated antibody comprising the HVR-H1, HVR-H2, and HVR-H3 of the heavy chain variable domains of monoclonal antibody 5C12 produced by the hybridoma cell line deposited with ATCC on May 15, 2013 having ATCC Accession Number PTA-120352, or progeny thereof. In some embodiments, the anti-C1s antibody is an isolated antibody comprising the HVR-L1, HVR-L2, and HVR-L3 of the light chain variable domains and the HVR-H1, HVR-H2, and HVR-H3 of the heavy chain variable domains of monoclonal antibody 5A1 produced by the hybridoma cell line deposited with ATCC on May 15, 2013 having ATCC Accession Number PTA-120352, or progeny thereof.

In some embodiments, the anti-C1s antibody inhibits the interaction between C1s and C1q. In some embodiments, the anti-C1s antibody inhibits the interaction between C1s and C1r. In some embodiments the anti-C1s antibody inhibits the interaction between C1s and C1q and between C1s and C1r. In some embodiments, the anti-C1s antibody inhibits the catalytic activity of C1s or the processing of pro-C1s to an active protease. In some embodiments, the anti-C1s antibody inhibits the interaction between C1s and its substrates such as C2 and C4. In some embodiments, the anti-C1s antibody binds to C1s respective interactions, at a stoichiometry of less than 2.5:1; 2.0:1; 1.5:1; or 1.0:1. In some embodiments, the anti-C1s antibody binds to C1s with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1s antibody binds C1s with a binding stoichiometry that ranges from 20:1 to 1.0:1 or less than 1.0:1. In certain embodiments, the anti-C1s antibody binds C1s with a binding stoichiometry that ranges from 6:1 to 1.0:1 or less than 1.0:1. In certain embodiments, the anti-C1s antibody binds C1s with a binding stoichiometry that ranges from 2.5:1 to 1.0:1 or less than 1.0:1. In preferred embodiments, the C1s antibody inhibits an interaction, such as the C1s-C4 interaction, at approximately equimolar concentrations of C1s and the anti-C1s antibody. In some embodiments the anti-C1s antibody inhibits activation of C1s, C1r, or of both C1s and C1r.

In some embodiments, the anti-C1s antibodies of this disclosure inhibit C1F hemolysis by at least 20%, at least 30%, at least 40%, at least 50%, at least 60%, at least 70%, at least 80%, at least 90%, at least 95%, or at least 99% relative to a control wherein the anti-C1s antibodies of this disclosure are absent (see also Example 3). In certain embodiments, anti-C1s antibodies of this disclosure inhibit C1F hemolysis by an amount that ranges from at least 30% to at least 99% relative to a control wherein the antibodies of this disclosure are absent. Methods for measuring C1F hemolysis are well known in the art (see also Example 3 for possible methods). The EC₅₀ values for antibodies of this disclosure with respect to C1F hemolysis may be less than less than 3 μg/ml; less than 2.5 μg/ml; less than 2.0 μg/ml; less than 1.5 μg/ml; less than 1.0 μg/ml; less than 0.5 μg/ml; less than 0.25 μg/ml; less than 0.1 μg/ml; or less than 0.05 μg/ml. Preferably, EC₅₀ values are less than 1.0 μg/ml (see also Example 3). Preferably, the anti-C1s antibodies of this disclosure inhibit C1F hemolysis at approximately equimolar concentrations of C1s and the respective anti-C1s antibody.

In some embodiments, the alternative pathway may amplify CDC initiated by C1q binding and subsequent C1s activation; in at least some of these embodiments, the anti-C1s antibodies of this disclosure inhibit the alternative pathway by at least 30%, at least 40%, at least 50%, at least 60%, at least 70%, at least 80%, at least 90%, at least 95%, or at least 99% relative to a control wherein the antibodies of this disclosure are absent. In certain embodiments, anti-C1s antibodies of this disclosure inhibit the alternative pathway by an amount that ranges from at least 30% to at least 99% relative to a control wherein the anti-C1s antibodies of this disclosure are absent.

In some embodiments, the anti-C1s antibodies of this disclosure prevent lesion formation in an ex vivo spinal cord slice model of NMO or in an in vivo mouse model of NMO. Methods for measuring lesion formation ex vivo or in vivo are well known in the art. Ex vivo lesion formation may be reduced at least by a relative score of 0.5, 1.0, 1.5, 2.0, 2.5, 3.0, 3.5, or 4.0. Preferably, ex vivo lesion formation is reduced by a relative score of at least 2.5. The EC₅₀ values for anti-C1s antibodies of this disclosure with respect to the prevention of ex vivo lesion formation may be less than 3 μg/ml; less than 2.5 μg/ml; less than 2.0 μg/ml; less than 1.5 μg/ml; less than 1.0 μg/ml; less than 0.5 μg/ml; less than 0.25 μg/ml; less than 0.1 μg/ml; or less than 0.05 μg/ml. Preferably, EC₅₀ values are less than 1.0 μM. In vivo lesion formation may be reduced by at least 5%, at least 10%, at least 15%, at least 20%, at least 35%, at least 40%, or at least 50% in terms of loss of staining (% of area). Staining may be assessed, without limitation, by AQP4 staining, GFAP staining, or MBP staining Preferably, in vivo lesion formation is reduced by at least 10%.

Anti-C1r Antibodies

The anti-C1r antibodies of this disclosure recognize and bind to complement factor C1r and/or C1r in the C1 complex of the classical complement activation pathway.

In some embodiments, the anti-C1r antibodies neutralize an activity of complement factor C1r. In some embodiments that anti-C1r antibody inhibits the interaction between C1r and C1q. In some embodiments, the anti-C1r antibody inhibits the interaction between C1r and C1s. In some embodiments, the anti-C1r antibody inhibits the interaction between C1r and C1q and between C1r and C1s. In some embodiments, the anti-C1r antibody inhibits the catalytic activity of C1r and/or the processing of pro-C1r to an active protease. In some embodiments, the anti-C1r antibody inhibits the respective interactions at a stoichiometry of less than 2.5:1; 2.0:1; 1.5:1; or 1.0:1. In some embodiments, the anti-C1r antibody binds to C1r with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1r antibody binds C1r with a binding stoichiometry that ranges from 20:1 to 1.0:1 or less than 1.0:1. In certain embodiments, the anti-C1r antibody binds C1r with a binding stoichiometry that ranges from 6:1 to 1.0:1 or less than 1.0:1. In certain embodiments, the anti-C1r antibody binds C1r with a binding stoichiometry that ranges from 2.5:1 to 1.0:1 or less than 1.0:1. In some embodiments the anti-C1r antibody inhibits activation of C1r, C1s, and/or both C1r and C1s. Activation of the serine proteases C1r and C1s can be measured, without limitation, by standard colorimetric or fluorescent serine protease assays or by a standard complement-mediated cell lysis assays that are well known in the art.

Anti-C1 Complex Antibodies

The anti-C1 complex antibodies of this disclosure recognize and bind to C1 complex and/or complement factors C1q, C1s, and/or C1r in the C1 complex of the classical complement activation pathway.

In some embodiments, the anti-C1 complex antibodies neutralize an activity of C1 complex, complement factor C1q, complement factor C1s, and/or complement factor C1r. In some embodiments the anti-C1 complex antibody inhibits the interaction between C1q and C1s, C1q and C1r, and/or C1s and C1r. In some embodiments, the anti-C1 complex antibody inhibits C1r or C1s activation or prevents their ability to act on C2 or C4. In some embodiments, the anti-C1 complex antibody inhibits the respective interactions at a stoichiometry of less than 2.5:1; 2.0:1; 1.5:1; or 1.0:1. In some embodiments, the anti-C1 complex antibody binds to the C1 complex with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1 complex antibody binds the C1 complex with a binding stoichiometry that ranges from 20:1 to 1.0:1 or less than 1.0:1. In certain embodiments, the anti-C1 complex antibody binds the C1 complex with a binding stoichiometry that ranges from 6:1 to 1.0:1 or less than 1.0:1. In certain embodiments, the anti-C1 complex antibody binds the C1 complex with a binding stoichiometry that ranges from 2.5:1 to 1.0:1 or less than 1.0:1.

Antibody Characteristics

Where antibodies of this disclosure inhibit the interaction between two or more complement factors, such as the interaction of C1q and C1s, or the interaction between factor C1q and an autoantibody, the interaction occurring in the presence of the antibody is reduced by at least 10%, at least 20%, at least 30%, at least 40%, at least 50%, at least 60%, at least 70%, at least 80%, at least 90%, at least 95%, at least 99%, or at least 99.9% compared to the interaction occurring in the absence of the antibody, but otherwise identical conditions. In certain embodiments, the interaction occurring in the presence of the antibody is reduced by an amount that ranges from at least 30% to at least 99.9%. Where antibodies of this disclosure inhibit activation of C1s and/or C1r, the serine protease activity of C1s and/or C1r in the presence of the antibody is reduced by at least 10%, at least 20%, at least 30%, at least 40%, at least 50%, at least 60%, at least 70%, at least 80%, at least 90%, at least 95%, at least 99%, or at least 99.9% compared to the serine protease activity in the absence of the antibody. In certain embodiments, the serine protease activity of C1s and/or C1r in the presence of the antibody in the presence of the antibody is reduced by an amount that ranges from at least 30% to at least 99.9%.

In some embodiments, the antibodies of this disclosure inhibit C4-cleavage by at least 20%, at least 30%, at least 40%, at least 50%, at least 60%, at least 70%, at least 80%, at least 90%, at least 95%, or at least 99% relative to a control wherein the antibodies of this disclosure are absent. In certain embodiments, the antibodies of this disclosure inhibit C4-cleavage by an amount that ranges from at least 30% to at least 99% relative to a control wherein the antibodies of this disclosure are absent. Methods for measuring C4-cleavage are well known in the art (see also Example 3 for possible methods). The EC₅₀ values for antibodies of this disclosure with respect C4-cleavage may be less than 3 μg/ml; less than 2.5 μg/ml; less than 2.0 μg/ml; less than 1.5 μg/ml; less than 1.0 μg/ml; less than 0.5 μg/ml; less than 0.25 μg/ml; less than 0.1 μg/ml; or less than 0.05 μg/ml. Preferably, EC₅₀ values are less than 1.0 μg/ml. Preferably, the antibodies of this disclosure inhibit C4-cleavage at approximately equimolar concentrations of C1s and the respective anti-C1s antibody.

In some embodiments, the antibodies of this disclosure inhibit autoantibody-dependent and complement-dependent cytotoxicity (CDC) by at least 10%, at least 20%, at least 30%, at least 40%, at least 50%, at least 60%, at least 70%, at least 80%, at least 90%, at least 95%, at least 99%, or at least 99.9% relative to a control wherein the antibodies of the present disclosure are absent. In certain embodiments, antibodies of the present disclosure inhibit CDC by an amount that ranges from at least 30% to at least 99.9%. The EC₅₀ values for antibodies of this disclosure, e.g., with respect to inhibition of autoantibody-dependent and complement-dependent cytotoxicity (CDC), may be less than 3 μg/ml; less than 2.5 μg/ml; 2.0 μg/ml; less than 1.5 μg/ml; less than 1.0 μg/ml; less than 0.5 μg/ml; less than 0.25 μg/ml; less than 0.1 μg/ml; or less than 0.05 μg/ml. Preferably, EC₅₀ values are less than 1.0 μg/ml (see also Example 3). In certain embodiments, the EC₅₀ values for antibodies of this disclosure range from 3 μg/ml to 0.05 μg/ml or less than 0.05 μg/ml. In some embodiments, the EC₅₀ values for antibodies of the present disclosure may be calculated in the presence of human complement, for example present in human serum. In some embodiments, the amount of human complement, for example human serum, ranges from less than 1% to at least 20%. In certain embodiments, the amount of human complement, for example human serum, is less than 1%. In certain embodiments, the amount of human complement, for example human serum, is at least 1%, at least 2%, at least 3%, at least 4% at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%.

In some embodiments, the antibodies of this disclosure inhibit complement-dependent cell-mediated cytotoxicity (CDCC) by at least 10%, at least 20%, at least 30%, at least 40%, at least 50%, at least 60%, at least 70%, at least 80%, at least 90%, at least 95%, at least 99%, or at least 99.9% relative to a control wherein the antibodies of the present disclosure are absent. In certain embodiments, antibodies of the present disclosure inhibit CDCC by an amount that ranges from at least 30% to at least 99.9%. Methods for measuring CDCC are well known in the art (see also Example 5 for possible methods). The EC₅₀ values for antibodies of this disclosure, for example, with respect CDCC inhibition may be less than 3 μg/ml; less than 2.5 μg/ml; 2.0 μg/ml; less than 1.5 μg/ml; less than 1.0 μg/ml; less than 0.5 μg/ml; less than 0.25 μg/ml; less than 0.1 μg/ml; or less than 0.05 μg/ml. Preferably, EC₅₀ values are less than 1.0 μg/ml. In certain embodiments, the EC₅₀ values for antibodies of the present disclosure range from 3 μg/ml to 0.05 μg/ml or less than 0.05 μg/ml. In some embodiments, the EC₅₀ values for antibodies of the present disclosure may be calculated in the presence of human complement, for example present in human serum. In some embodiments, the amount of human complement, for example human serum, ranges from less than 1% to at least 20%. In certain embodiments, the amount of human complement, for example human serum, is less than 1%. In certain embodiments, the amount of human complement, for example human serum, is at least 1%, at least 2%, at least 3%, at least 4% at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%. In preferred embodiments, the antibodies of the present disclosure inhibit CDCC but not antibody-dependent cellular cytotoxicity (ADCC; see also Example 5). In some embodiments, the antibodies of the present disclosure do not inhibit the lectin complement activation pathway.

As disclosed herein, the alternative pathway may amplify CDC initiated by C1 complex, e.g., by C1q binding to an autoantibody such as anti-GQ1b autoantibodies. Accordingly, in some of embodiments, the antibodies of the present disclosure may inhibit the alternative pathway by at least 10%, at least 20%, at least 30%, at least 40%, at least 50%, at least 60%, at least 70%, at least 80%, at least 90%, at least 95%, at least 99%, or at least 99.9% relative to a control wherein the antibodies of the present disclosure are absent (see also Example 5). In certain embodiments, antibodies of the present disclosure inhibit the alternative pathway by an amount that ranges from at least 30% to at least 99.9%.

Additional anti-C1q, anti-C1s, anti-C1r and/or anti-C1 complex antibodies, e.g., antibodies that specifically bind to a C1q, C1s, or C1r protein, or the C1 complex of the present disclosure, may be identified, screened, and/or characterized for their physical/chemical properties and/or biological activities by various assays known in the art.

Additional anti-C1q, anti-C1s, anti-C1r, or anti-C1 complex antibodies, e.g., antibodies that specifically bind to a C1q, C1s, or C1r protein, or the C1 complex of the present disclosure, may be identified, screened, and/or characterized for their physical/chemical properties and/or biological activities by various assays known in the art.

In some embodiments, the present disclosure provides anti-C1q, anti-C1s, anti-C1r and/or anti-C1 complex antibodies. The antibodies of this disclosure may have one or more of the following characteristics. The antibodies of this disclosure may be polyclonal antibodies, monoclonal antibodies, human antibodies, humanized antibodies, chimeric antibodies, antibody fragments (e.g., Fab, Fab′-SH, Fv, scFv, and (Fab′)₂ fragments), bispecific and polyspecific antibodies, multivalent antibodies, and heteroconjugate antibodies. The antibodies of this disclosure may further contain engineered effector functions, amino acid sequence modifications or other antibody modifications known in the art; e.g., the constant region of the anti-C1q, anti-C1s, C1r and/or anti-C1 complex antibodies described herein may be modified to impair complement activation.

In certain embodiment, antibodies of the present disclosure are bispecific antibodies recognizing a first antigen and a second antigen. In some embodiments, the first antigen is a C1q antigen, a C1s antigen, a C1r antigen, and/or a C1 complex antigen. In some embodiments, the second antigen is an antigen facilitating transport across the blood-brain-barrier, including without limitation, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005.

In some embodiments, the antibodies of this disclosure prevent GBS, or one or more symptoms of GBS. In certain embodiments, prevention of GBS or one or more symptoms of GBS by the antibodies of the present disclosure is measure by inhibition of C3c deposition, inhibition of MAC depiction, inhibition of axonal damage formation, and/or inhibition of respiratory muscle damage in an ex vivo model of GBS, or by an in vivo mouse model of GBS. In some embodiments, the antibodies of this disclosure inhibit C3c deposition, MAC deposition, axonal damage formation, and/or respiratory muscle damage in an ex vivo model of GBS or in an in vivo mouse model of GBS. Methods for measuring C3c deposition, MAC deposition, axonal damage formation, and/or respiratory muscle damage ex vivo or in vivo are well known in the art (see also Examples 7-9 for exemplary methods).

Additional anti-C1q, anti-C1s, anti-C1r and anti-C1 complex antibodies, e.g., antibodies that specifically bind to a C1q protein, a C1s protein, a C1r protein, or a C1 complex, of the present disclosure, may be identified, screened, and/or characterized for their physical/chemical properties and/or biological activities by various assays known in the art.

### Antibody Preparation

Anti-C1q, anti-C1s, anti-C1r, and/or anti-C1 complex antibodies of the present disclosure can encompass polyclonal antibodies, monoclonal antibodies, humanized antibodies, chimeric antibodies, human antibodies, antibody fragments (e.g., Fab, Fab′-SH, Fv, scFv, and F(ab′)₂), bispecific and polyspecific antibodies, multivalent antibodies, heteroconjugate antibodies, library derived antibodies, antibodies having modified effector functions, fusion proteins containing an antibody portion, and any other modified configuration of the immunoglobulin molecule that includes an antigen recognition site, such as an epitope having amino acid residues of a C1q, C1s, or C1r protein, or the C1 complex of the present disclosure, including glycosylation variants of antibodies, amino acid sequence variants of antibodies, and covalently modified antibodies. The anti-C1q, anti-C1s, anti-C1r, and/or anti-C1 complex antibodies may be human, murine, rat, or of any other origin (including chimeric or humanized antibodies).

(1) Polyclonal Antibodies

Polyclonal antibodies, such as polyclonal anti-C1q, anti-C1s, anti-C1r, and/or anti-C1 complex antibodies, are generally raised in animals by multiple subcutaneous (sc) or intraperitoneal (ip) injections of the relevant antigen and an adjuvant. It may be useful to conjugate the relevant antigen (e.g., a purified or recombinant C1q, C1s, or C1r protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies, such as monoclonal anti-C1q, anti-C1s, anti-C1r, and/or anti-C1 complex antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal anti-C1q, anti-C1s, anti-C1r, and/or anti-C1 complex antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant C1q, C1s, or C1r protein of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant C1q, C1s, or C1r protein of the present disclosure) or a fusion variant thereof. Generally peripheral blood lymphocytes (“PBLs”) are used if cells of human origin are desired, while spleen or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient-cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif. USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a C1q, C1s, or C1r protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a C1q, C1s, or C1r protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-C1q, anti-C1s, anti-C1r, and/or anti-C1 complex monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, anti-C1q, anti-C1s, anti-C1r, and/or anti-C1 complex antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind a C1q, C1s, or C1r protein of the present disclosure).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-C1q, anti-C1s, anti-C1r, and/or anti-C1 complex antibodies of the present disclosure or fragments thereof) may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid anti-C1q, anti-C1s, anti-C1r, and/or anti-C1 complex antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

(3) Humanized Antibodies

Anti-C1q, anti-C1s, anti-C1r, and/or anti-C1 complex antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human anti-C1q, anti-C1s, anti-C1r, and/or anti-C1 complex antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., C1q, C1s, or C1r proteins of the present disclosure), is achieved. In general, the CDR residues are directly and most substantially involved in influencing antigen binding.

Various forms of the humanized anti-C1q, anti-C1s, anti-C1r, and/or anti-C1 complex antibody are contemplated. For example, the humanized anti-C1q, anti-C1s, anti-C1r, and/or anti-C1 complex antibody may be an antibody fragment, such as an Fab, which is optionally conjugated with one or more cytotoxic agent(s) in order to generate an immunoconjugate. Alternatively, the humanized anti-C1q, anti-C1s, anti-C1r, and/or anti-C1 complex antibody may be an intact antibody, such as an intact IgG1 antibody.

(4) Human Antibodies

Alternatively, human anti-C1q, anti-C1s, anti-C1r, and/or anti-C1 complex antibodies can be generated. For example, it is now possible to produce transgenic animals (e.g., mice) that are capable, upon immunization, of producing a full repertoire of human antibodies in the absence of endogenous immunoglobulin production. The homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Nat'l Acad. Sci. USA, 90:2551 (1993); Jakobovits et al., Nature, 362:255-258 (1993); Bruggermann et al., Year in Immunol., 7:33 (1993); U.S. Pat. No. 5,591,669 and WO 97/17852.

Alternatively, phage display technology can be used to produce human anti-C1q, anti-C1s, anti-C1r, and/or anti-C1 complex antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also U.S. Pat. Nos. 5,565,332 and 5,573,905. Additionally, yeast display technology can be used to produce human anti-C1q, anti-C1s, anti-C1r, and/or anti-C1 complex antibodies and antibody fragments in vitro (e.g., WO 2009/036379; WO 2010/105256; WO 2012/009568; US 2009/0181855; US 2010/0056386; and Feldhaus and Siegel (2004) J. Immunological Methods 290:69-80). In other embodiments, ribosome display technology can be used to produce human anti-C1q, anti-C1s, anti-C1r, and/or anti-C1 complex antibodies and antibody fragments in vitro (e.g., Roberts and Szostak (1997) Proc Natl Acad Sci 94:12297-12302; Schaffitzel et al. (1999) J. Immunolical Methods 231:119-135; Lipovsek and Plückthun (2004) J. Immunological Methods 290:51-67).

The techniques of Cole et al., and Boerner et al., are also available for the preparation of human anti-C1q, anti-C1s, anti-C1r, and/or anti-C1 complex monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human anti-C1q, anti-C1s, anti-C1r, and/or anti-C1 complex antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human anti-C1q, anti-C1s, anti-C1r, and/or anti-C1 complex antibodies may also be generated in vitro by activated B-cells (see U.S. Pat. Nos. 5,567,610 and 5,229,275).

(5) Antibody Fragments

In certain embodiments there are advantages to using anti-C1q, anti-C1s, anti-C1r, and/or anti-C1 complex antibody fragments, rather than whole anti-C1q, anti-C1s, anti-C1r, and/or anti-C1 complex antibodies. Smaller fragment sizes allow for rapid clearance.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding anti-C1q, anti-C1s, anti-C1r, and/or anti-C1 complex antibodies of the present disclosure. Fab, Fv and scFv antibody fragments can all be expressed in and secreted from E. coli, thus allowing the straightforward production of large amounts of these fragments. Anti-C1q, anti-C1s, anti-C1r, and/or anti-C1 complex antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. No. 5,571,894 and U.S. Pat. No. 5,587,458. The anti-C1q, anti-C1s, anti-C1r, and/or anti-C1 complex antibody fragment may also be a “linear antibody,” e.g., as described in U.S. Pat. No. 5,641,870. Such linear antibody fragments may be monospecific or bispecific.

(6) Bispecific and Polyspecific Antibodies

Bispecific antibodies (BsAbs) are antibodies that have binding specificities for at least two different epitopes, including those on the same or another protein (e.g., one or more C1q, C1s, or C1r proteins of the present disclosure). Alternatively, one part of a BsAb can be armed to bind to the target C1q, C1s, or C1r antigen, and another can be combined with an arm that binds to a second protein. Such antibodies can be derived from full length antibodies or antibody fragments (e.g., F(ab′)₂ bispecific antibodies).

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986).

According to another approach described in WO 96/27011 or U.S. Pat. No. 5,731,168, the interface between a pair of antibody molecules can be engineered to maximize the percentage of heterodimers which are recovered from recombinant-cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T-cells, as well as trigger the lytic activity of human cytotoxic lymphocytes against human breast tumor targets.

Various techniques for making and isolating bivalent antibody fragments directly from recombinant-cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J. Immunol. 147:60 (1991).

Exemplary bispecific antibodies may bind to two different antigens. In some embodiments a bispecific antibody binds to a first antigen C1q, C1r, or C1s and a second antigen facilitating transport across the blood-brain barrier. Numerous antigens are known in the art that facilitate transport across the blood-brain barrier (see, e.g., Gabathuler R., Approaches to transport therapeutic drugs across the blood-brain barrier to treat brain diseases, Neurobiol. Dis. 37 (2010) 48-57). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, and Angiopep peptides such as ANG1005 (see, e.g., Gabathuler, 2010).

(7) Multivalent Antibodies

A multivalent antibody may be internalized (and/or catabolized) faster than a bivalent antibody by a cell expressing an antigen to which the antibodies bind. The anti-C1q, anti-C1s, anti-C1r, and/or anti-C1 complex antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

(8) Heteroconjugate Antibodies

Heteroconjugate antibodies are also within the scope of the present disclosure.

Heteroconjugate antibodies are composed of two covalently joined antibodies (e.g., anti-C1q, anti-C1s, anti-C1r, and/or anti-C1 complex antibodies of the present disclosure or antibody fragments thereof). For example, one of the antibodies in the heteroconjugate can be coupled to avidin, the other to biotin. Such antibodies have, for example, been proposed to target immune system cells to unwanted cells, U.S. Pat. No. 4,676,980, and have been used to treat HIV infection. International Publication Nos. WO 91/00360, WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

(9) Effector Function Engineering

It may also be desirable to modify an anti-C1q, anti-C1s, anti-C1r, and/or anti-C1 complex antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000).

The constant region of the anti-complement antibodies described herein may also be modified to impair complement activation. For example, complement activation of IgG antibodies following binding of the C1 component of complement may be reduced by mutating amino acid residues in the constant region in a C1 binding motif (e.g., C1q binding motif). It has been reported that Ala mutation for each of D270, K322, P329, P331 of human IgG1 significantly reduced the ability of the antibody to bind to C1q and activating complement. For murine IgG2b, C1s binding motif constitutes residues E318, K320, and K322. Idusogie et al. (2000) J. Immunology 164:4178-4184; Duncan et al. (1988) Nature 322: 738-740. As the C1q binding motif E318, K320, and K322 identified for murine IgG2b is believed to be common for other antibody isotypes (Duncan et al. (1988) Nature 322:738-740), C1q, C1s, C1r, or C1 complex binding activity for IgG2b can be abolished by replacing any one of the three specified residues with a residue having an inappropriate functionality on its side chain. It is not necessary to replace the ionic residues only with Ala to abolish C1q, C1s, C1r, or C1 complex binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish C1q, C1s, C1r, or C1 complex binding. In addition, it is also possible to use such polar non-ionic residues as Ser, Thr, Cys, and Met in place of residues 320 and 322, but not 318, in order to abolish C1q, C1s, C1r, or C1 complex binding activity. In addition, removal of carbohydrate modifications of the Fc region necessary for complement binding can prevent complement activation Glycosylation of a conserved asparagine (Asn-297) on the CH2 domain of IgG heavy chains is essential for antibody effector functions (Jefferis et al. (1998) Immunol Rev 163:59-76). Modification of the Fc glycan alters IgG conformation and reduces the Fc affinity for binding of complement protein C1q, C1s, or C1r and effector cell receptor FcR (Alhorn et al. (2008) PLos ONE 2008; 3:e1413). Complete removal of the Fc glycan abolishes CDC and ADCC. Deglycosylation can be performed using glycosidase enzymes for example Endoglycosidase S (EndoS), a 108 kDa enzyme encoded by the gene endoS of Streptococcus pyogenes that selectively digests asparagine-linked glycans on the heavy chain of all IgG subclasses, without action on other immunoglobulin classes or other glycoproteins (Collin et al. (2001) EMBO J 2001; 20:3046-3055).

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(10) Other Amino Acid Sequence Modifications

Amino acid sequence modifications of anti-C1q, anti-C1s, anti-C1r, and/or anti-C1 complex antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a C1q, C1s, or C1r protein of the present disclosure). The amino acid changes also may alter post-translational processes of the antibody, such as changing the number or position of glycosylation sites.

A useful method for identification of certain residues or regions of the anti-C1q, anti-C1s, anti-C1r, and/or anti-C1 complex antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino-(“N”) and/or carboxy-(“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table A below under the heading of “preferred substitutions”. If such substitutions result in a change in biological activity, then more substantial changes, denominated “exemplary substitutions” in Table A, or as further described below in reference to amino acid classes, may be introduced and the products screened.

TABLE A
Amino Acid Substitutions

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-C1q, anti-C1s, anti-C1r, and/or anti-C1 complex antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a C1q, C1s, or C1r protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibodies (e.g., an anti-C1q, anti-C1s, anti-C1r, and/or anti-C1 complex antibody of the present disclosure) or antibody fragments.

(11) Other Antibody Modifications

Anti-C1q, anti-C1s, anti-C1r, and/or anti-C1 complex antibodies of the present disclosure, or antibody fragments thereof, can be further modified to contain additional non-proteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

### Nucleic Acids, Vectors, and Host Cells

Anti-C1q, anti-C1s, anti-C1r, and/or anti-C1 complex antibodies of the present disclosure may be produced using recombinant methods and compositions, e.g., as described in U.S. Pat. No. 4,816,567. In some embodiments, isolated nucleic acids having a nucleotide sequence encoding any of the anti-C1q, anti-C1s, anti-C1r, and/or anti-C1 complex antibodies of the present disclosure are provided. Such nucleic acids may encode an amino acid sequence containing the VL and/or an amino acid sequence containing the VH of the anti-C1q, anti-C1s, anti-C1r, and/or anti-C1 complex antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell).

Methods of making an anti-C1q, anti-C1s, anti-C1r, and/or anti-C1 complex antibody of the present disclosure are provided. In some embodiments, the method includes culturing a host cell of the present disclosure containing a nucleic acid encoding the anti-C1q, anti-C1s, anti-C1r, and/or anti-C1 complex antibody, under conditions suitable for expression of the antibody. In some embodiments, the antibody is subsequently recovered from the host cell (or host cell culture medium).

For recombinant production of an anti-C1q, anti-C1s, anti-C1r, and/or anti-C1 complex antibody of the present disclosure, a nucleic acid encoding the anti-C1q, anti-C1s, anti-C1r, and/or anti-C1 complex antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-C1q, anti-C1s, anti-C1r, and/or anti-C1 complex antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-C1q, anti-C1s, anti-C1r, and/or anti-C1 complex antibody of the present disclosure.

Suitable host cells for cloning or expression of antibody-encoding vectors include prokaryotic or eukaryotic cells. For example, anti-C1q, anti-C1s, anti-C1r, and/or anti-C1 complex antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B.K.C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants.).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Pharmaceutical Compositions

Anti-C1q, anti-C1s, anti-C1r, and/or anti-C1 complex antibodies of the present disclosure can be incorporated into a variety of formulations for therapeutic use (e.g., by administration) or in the manufacture of a medicament (e.g., for treating or preventing an autoimmune or neurodegenerative disease) by combining the antibodies with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody, such as an anti-C1q, anti-C1s, anti-C1r, and/or anti-C1 complex antibody of the present disclosure, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical Dosages

Pharmaceutical compositions of the present disclosure containing an anti-C1q, anti-C1s, anti-C1r, and/or anti-C1 complex antibody of the present disclosure may be used (e.g., administered to an individual in need of treatment with anti-C1q, anti-C1s, anti-C1r, and/or anti-C1 complex antibody, preferably a human), in accordance with known methods, such as intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the anti-C1q, anti-C1s, anti-C1r, and/or anti-C1 complex antibodies of the present disclosure, normal dosage amounts may vary from about 10 ng/kg up to about 100 mg/kg of an individual's body weight or more per day, preferably about 1 mg/kg/day to 10 mg/kg/day, depending upon the route of administration. For repeated administrations over several days or longer, depending on the severity of the disease, disorder, or condition to be treated, the treatment is sustained until a desired suppression of symptoms is achieved.

An exemplary dosing regimen may include administering an initial dose of an anti-C1q, anti-C1s, anti-C1r, and/or anti-C1 complex antibody, of about 2 mg/kg, followed by a weekly maintenance dose of about 1 mg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the anti-C1q, anti-C1s, anti-C1r, and/or anti-C1 complex antibody administered, can vary over time independently of the dose used.

Dosages for a particular anti-C1q, anti-C1s, anti-C1r, and/or anti-C1 complex antibody may be determined empirically in individuals who have been given one or more administrations of the anti-C1q, anti-C1s, anti-C1r, and/or anti-C1 complex antibody. Individuals are given incremental doses of an anti-C1q, anti-C1s, anti-C1r, and/or anti-C1 complex antibody. To assess efficacy of an anti-C1q, anti-C1s, anti-C1r, and/or anti-C1 complex antibody, any clinical symptom of, for example GBS, can be monitored.

Administration of an anti-C1q, anti-C1s, anti-C1r, and/or anti-C1 complex antibody of the present disclosure can be continuous or intermittent, depending, for example, on the recipient's physiological condition, whether the purpose of the administration is therapeutic or prophylactic, and other factors known to skilled practitioners. The administration of an anti-C1q, anti-C1s, anti-C1r, and/or anti-C1 complex antibody may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the invention that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Therapeutic Uses

The present disclosure provides anti-C1q, anti-C1s, anti-C1r, and/or anti-C1 complex antibodies which can bind to and neutralize a biologic activity of C1q, C1s, C1r, and/or C1 complex. These anti-C1q, anti-C1s, anti-C1r, and/or anti-C1 complex antibodies are useful for preventing, reducing risk, or treating a wide range of autoimmune, such as GBS. Accordingly, as disclosed herein, anti-C1q, anti-C1s, anti-C1r, and/or anti-C1 complex antibodies of the present disclosure may be used for treating, preventing, or reducing risk of GBS in an individual. In some embodiments, the individual has GBS. In some embodiments, the individual is a human.

As disclosed herein, “Guillain-Barré syndrome,” “GBS,” “Landry's paralysis,” and “Guillain-Barr\acute{w}-Strohl syndrome” may be used interchangeably and refer to a disorder in which the body's immune system attacks part of the peripheral nervous system. The exact cause of Guillain-Barre syndrome is unknown, but it is often preceded by an infectious illness, such as a respiratory infection or the stomach flu.

The first symptoms of this disorder include varying degrees of weakness or legs that tend to buckle, with or without tingling sensations in the legs. In many instances the symmetrical weakness and abnormal sensations spread, usually over periods of hours to days, to the arms, upper body, and facial muscles. Frequently, the lower cranial nerves may be affected, leading to bulbar weakness, oropharyngeal dysphagia (drooling, or difficulty swallowing and/or maintaining an open airway) and respiratory difficulties, and at times interference with blood pressure or heart rate. Most patients require hospitalization and about 30% require ventilatory assistance for treatment of type II respiratory failure. If present, sensory loss usually manifests as loss of proprioception (position sense) and areflexia (complete loss of deep tendon reflexes), which is an important feature of GBS. Loss of pain and temperature sensation is usually mild, as pain is a common symptom in GBS, presenting as deep aching pain, usually in the weakened muscles. These pains are self-limited and may be treated with standard analgesics. Bladder dysfunction may also occur in severe cases.

Accordingly, the antibodies of the present disclosure may be used to treat, prevent, or improve one of more symptoms of GBS. In some embodiments, the present disclosure provides methods of treating, preventing, or improving one or more symptoms in individuals having GBS, by administering an anti-C1q, anti-C1s, anti-C1r, and/or anti-C1 complex antibody of the present disclosure to, for example, inhibit the interaction between C1q and an autoantibody, such as an anti-ganglioside autoantibody, the interaction of C1q and C1r, and/or the interaction of C1q and C1s. In some embodiments, the anti-C1q, anti-C1s, anti-C1r, and/or anti-C1 complex antibody inhibits C3c deposition. In some embodiments, the anti-C1q, anti-C1s, anti-C1r, and/or anti-C1 complex antibody inhibits membrane attack complex (MAC) deposition. In some embodiments, the anti-C1q, anti-C1s, anti-C1r, and/or anti-C1 complex antibody inhibits axonal damage formation. In some embodiments, the anti-C1q, anti-C1s, anti-C1r, and/or anti-C1 complex antibody inhibits respiratory muscle damage. The anti-C1q, anti-C1s, anti-C1r, and/or anti-C1 complex antibody may be administered to cells in vitro to prevent complement dependent cytotoxicity or complement-dependent cell-mediated cytotoxicity. The anti-C1q, anti-C1s, anti-C1r, and/or anti-C1 complex antibody may also be administered ex vivo in whole-mount muscle models of GBS. Alternatively, the anti-C1q, anti-C1s, anti-C1r, and/or anti-C1 complex antibody may be administered in vivo (e.g., by administering the antibody to an individual, such as a murine or human individual) to prevent to prevent C3c deposition or axonal damage formation.

### Combination Treatments

The antibodies of the present disclosure may be used, without limitation, in combination with any additional treatment for autoimmune and/or neurodegenerative diseases, such as GBS, including, without limitation, immunosuppressive therapies.

In some embodiments, an anti-C1q, anti-C1s, anti-C1r, and/or anti-C1 complex antibody of this disclosure is administered in therapeutically effective amounts in combination with a second neutralizing anti-complement factor antibody, such as an anti-C1q, anti-C1s, anti-C1r, and/or anti-C1 complex antibody, or a second anti-C1q, anti-C1s, anti-C1r, and/or anti-C1 complex antibody. In some embodiments, an anti-C1q, anti-C1s, anti-C1r, and/or anti-C1 complex antibody of this disclosure is administered in therapeutically effective amounts with a second and a third neutralizing anti-complement factor antibody, such as a second anti-C1q, anti-C1s, anti-C1r, and/or anti-C1 complex antibody.

In some embodiments, the anti-C1q, anti-C1s, anti-C1r, and/or anti-C1 complex antibodies of this disclosure are administered in combination with an inhibitor of antibody-dependent cellular cytotoxicity (ADCC). ADCC inhibitors may include, without limitation, soluble NK cell inhibitory receptors such as the killer cell Ig-like receptors (KIRs), which recognize HLA-A, HLA-B, or HLA-C and C-type lectin CD94/NKG2A heterodimers, which recognize HLA-E (see, e.g., López-Botet M., T. Bellón, M. Llano, F. Navarro, P. Garcia & M. de Miguel. (2000), Paired inhibitory and triggering NK cell receptors for HLA class I molecules. Hum. Immunol. 61: 7-17; Lanier L. L. (1998) Follow the leader: NK cell receptors for classical and nonclassical MHC class I. Cell 92: 705-707.), and cadmium (see, e.g., Immunopharmacology 1990; Volume 20, Pages 73-8).

In some embodiments, the antibodies of this disclosure are administered in combination with an inhibitor of the alternative pathway of complement activation. Such inhibitors may include, without limitation, factor B blocking antibodies, factor D blocking antibodies, soluble, membrane-bound, tagged or fusion-protein forms of CD59, DAF, CR1, CR2, Crry or Comstatin-like peptides that block the cleavage of C3, non-peptide C3aR antagonists such as SB 290157, Cobra venom factor or non-specific complement inhibitors such as nafamostat mesilate (FUTHAN; FUT-175), aprotinin, K-76 monocarboxylic acid (MX-1) and heparin (see, e.g., T. E. Mollnes & M. Kirschfink, Molecular Immunology 43 (2006) 107-121). In some embodiments, the antibodies of this disclosure are administered in combination with an inhibitor of the interaction between the autoantibody and its autoantigen. Such inhibitors may include purified soluble forms of the autoantigen, or antigen mimetics such as peptide or RNA-derived mimotopes, including mimotopes of the AQP4 antigen. Alternatively, such inhibitors may include blocking agents that recognize the autoantigen and prevent binding of the autoantibody without triggering the classical complement pathway. Such blocking agents may include, e.g., autoantigen-binding RNA aptamers or antibodies lacking functional C1q binding sites in their Fc domains (e.g., Fab fragments or antibody otherwise engineered not to bind C1q).

### Kits

The invention also provides kits containing antibodies of this disclosure for use in the methods of the present disclosure. Kits of the invention may include one or more containers comprising a purified anti-C1q, anti-C1s, anti-C1r, and/or anti-C1 complex antibody and instructions for use in accordance with methods known in the art. Generally, these instructions comprise a description of administration of the inhibitor to treat or diagnose, e.g., GBS (such as an antibody of this disclosure), according to any methods known in the art. The kit may further comprise a description of selecting an individual suitable for treatment based on identifying whether that individual has the disease and the stage of the disease.

The instructions generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating GBS. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an inhibitor of classical complement pathway. The container may further comprise a second pharmaceutically active agent.

Kits may optionally provide additional components such as buffers and interpretive information. Normally, the kit comprises a container and a label or package insert(s) on or associated with the container.

### Diagnostic Uses

The antibodies of this disclosure also have diagnostic utility. This disclosure therefore provides for methods of using the antibodies of this disclosure for diagnostic purposes, including the detection of C1q, C1s, C1r, and/or C1 complex in tissues, including tissues of a human patient. The anti-C1q, anti-C1s, anti-C1r, and/or anti-C1 complex antibodies of this disclosure are further useful for detecting synapses and synapse loss, e.g., synapse loss experienced by patients suffering from GBS. The phenomenon of synapse loss in neurodegeneration is well understood in the art. See, e.g., U.S. Patent Publication Nos. 2012/0195880 and 2012/0328601.

In some embodiments, the diagnostic methods involve the administration of an anti-C1q, anti-C1s, anti-C1r, and/or anti-C1 complex antibody of this disclosure to an individual and the detection of antibody levels bound to the synapses of the individual. Antibody binding to the synapses of an individual can be quantitatively measured by non-invasive techniques such as positron emission tomography (PET), X-ray computed tomography, single-photon emission computed tomography (SPECT), computed tomography (CT), and computed axial tomography (CAT).

In some embodiments, the diagnostic methods involve detecting synapses in a biological sample, such as a biopsy specimen, a tissue, or a cell. An anti-C1q, anti-C1s, anti-C1r, and/or anti-C1 complex antibody is contacted with the biological sample and the level of antibody bound to the synapses present in the biological sample is then detected. The detection can be quantitative. Antibody detection in biological samples may occur with any method known in the art, including immunofluorescence microscopy, immunocytochemistry, immunohistochemistry, ELISA, FACS analysis or immunoprecipitation.

Quantitation of synapse-bound antibodies provides a relative quantitative measure for the number of synapses present in the individual. The diagnostic methods are typically repeated on a regular basis, whereas the exact periodicity of the diagnostic measurement depends on many factors, including the nature of the neurodegenerative disease, the stage of disease progression, treatment modalities and many other factors. Repeat diagnostic measurements typically reveal progressive synapse loss in patients suffering from neurodegenerative diseases. Synapse loss may be followed over time in individual patients suffering from neurodegenerative diseases, but it may also be determined in a diseased patient population relative to a healthy patient population at any single point in time. Where patients are undergoing a specific therapy, the relative loss of synapse numbers in individuals undergoing the specific therapy relative to the synapse loss observed in patients not undergoing any treatments or undergoing control treatments can be used to assess the efficacy of the specific therapy provided.

The invention will be more fully understood by reference to the following Examples. They should not, however, be construed as limiting the scope of the invention. All citations throughout the disclosure are hereby expressly incorporated by reference.

### EXAMPLES

### Example 1

### Production of Anti-C1s Antibodies

The anti-C1s antibodies of this disclosure, including 5A1 and 5C12 (also referred to as C1s^{mAb1} and C1s^{mAb2}), were generated by immunizing mice with human C1s enzyme purified from human plasma (Complement Technology Inc. Tyler Tex., Cat # A103) using standard mouse immunization and hybridoma screening technologies (Milstein, C (1999). Bioessays 21: 966-73; Mark Page, Robin Thorpe, The Protein Protocols Handbook 2002, Editors: John M. Walker, pp 1111-1113). In brief, female BALB/c mice were injected intraperitoneal with 25 μg of protein in complete Freund's adjuvant (CFA) on Day 0 and boosts were done with 25 μg of C1s enzyme in incomplete Freund's adjuvant (IFA) on days 21, 42, 52, and a final intravenous boost on day 63. Four days following the final boost the mice were euthanized, spleens removed, and splenocytes were fused with the myeloma cell line SP2/0. Fused cells were grown on hypoxanthine-aminopterin-thymidine (HAT) selective semi-solid media for 10-12 days and the resulting hybridomas clones were transferred to 96-well tissue culture plates and grown in HAT medium until the antibody titre is high. The antibody rich supernatants of the clones were isolated and tested in an ELISA assay for reactivity with C1s. Positive clones were isotyped and cultured for 32 days (post HAT selection) to identify stable expressing clones.

Hybridoma cell lines producing anti-C1s antibodies 5A1 and 5C12 were deposited with ATCC on May 15, 2013 having ATCC Accession Numbers PTA-120351 and PTA-120352. The anti-C1s antibodies 5A1 and 5C12 were shown to bind to C1s and C1s-Pro and to neutralize biological functions of C1s in cellular and biochemical assays (see, e.g., Examples 2-4).

### Example 2

### Anti-C1s Antibodies Specifically Bind to C1s and C1s-Pro

First, anti-C1s antibodies were screened for C1s and C1s proenzyme binding by ELISA.

ELISA assays were conducted using standard protocols. Briefly, the assays were conducted as follows. The day before the assay was performed, 96-well microtiter plates were coated at 0.2 μg/well of C1s-enzyme antigen in 100 μL/well carbonate coating buffer pH9.6 overnight at 4° C. Next, the plates were blocked with 3% milk powder in PBS for 1 hour at room temperature. Next, hybridoma tissue culture supernatants were plated at 100 μL/well for 1 hour at 37° C. with shaking. The secondary antibody (1:10,000 goat anti-mouse IgG/IgM(H+L)-HRP) was applied at 100 μL/well for 1.5 hours at room temperature with shaking TMB substrate was added at 50 μL/well for 5 minutes at room temperature in the dark. The reaction stopped with 50 μL/well 1M HCl and read at 450 nm.

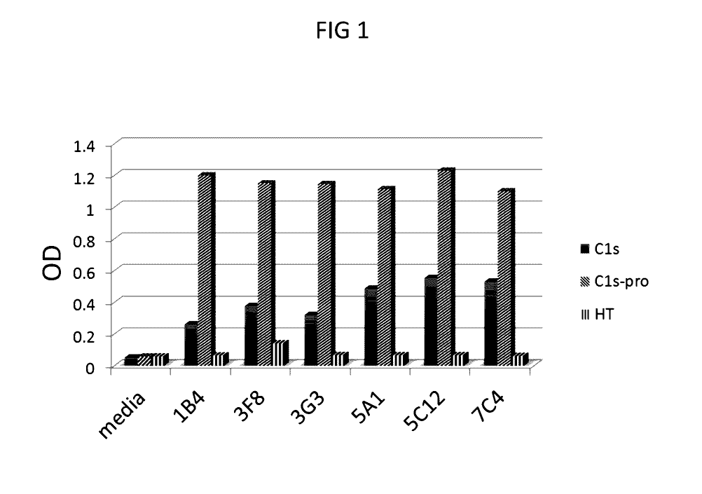

Six hybridoma supernatants (from 1B4, 3F8, 3G3, 5A1, 5C12, and 7C4) were tested for C1s and C1s proenzyme binding (FIG. 1). All six supernatants showed strong binding signals for the C1s proenzyme (middle column) as well as the mature C1s protease (left column). Only background binding signals were observed with the negative control protein, human transferrin (HT, right column) (FIG. 1).

These results showed that the antibodies produced by the hybridoma cells 1B4, 3F8, 3G3, 5A1, 5C12, and 7C4 specifically bind C1s and the C1s proenzyme.

### Example 3

### Anti-C1s Antibodies Inhibit Complement-Mediated Hemolysis

Next, the ability of anti-C1s antibodies to neutralize cellular activities of C1s was tested in a complement hemolytic assay.

A modified CH50 assay (also referred to as C1F hemolysis assay) was performed that provided limiting quantities of the C1 complex from human serum to provide greater sensitivity for assessing C1 activity and potential C1 inhibition. In brief, the assay was conducted as follows. First, 3×10⁷ sheep red blood cells (RBC) were incubated with anti-sheep RBC IgM antibody to generate activated erythrocytes (EA cells). The EA cells were then incubated with purified C4b protein to create EAC4b cells. EAC4b cells were subsequently incubated with diluted (1:1000-1:10000) normal human serum (NHS) that was pre-incubated with or without anti-C1s and control mouse IgG antibodies, to provide a limiting quantity of human C1. Next, the resulting EAC14 cells were incubated with purified human C2 protein to generate EAC14b2a cells. Finally, guinea pig serum was added in an EDTA buffer and incubated at 37° C. for 30 minutes. Cell lysis was measured in a spectrophotometer at 450 nm.

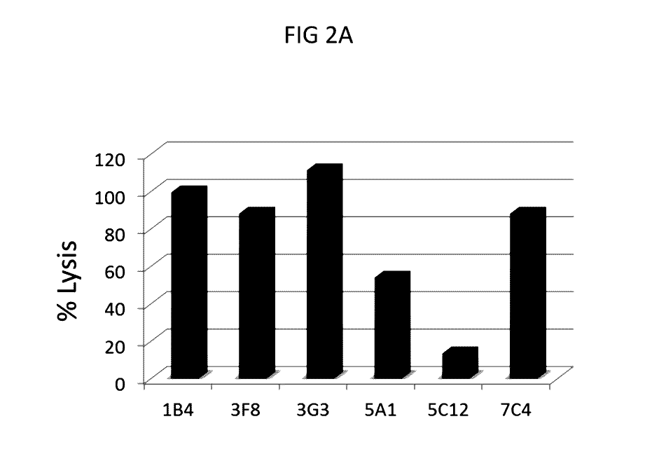

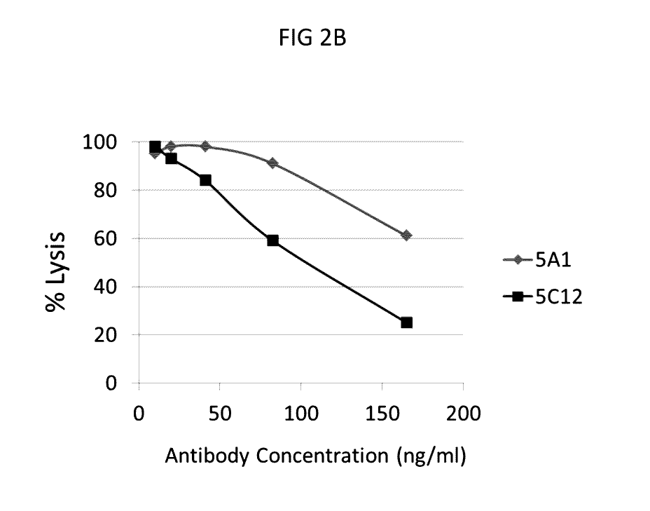

Six C1s and C1s proenzyme binding antibodies (1B4, 3F8, 3G3, 5A1, 5C12, and 7C4) were tested for their ability to suppress complement mediated hemolysis. Initial testing was conducted at a single antibody concentration (165 ng/ml). Out of the six antibodies tested, only 5A1 and 5C12 antibodies showed substantial inhibition of hemolysis, whereas 1B4, 3F8, 3G3, and 7C4 were essentially inactive (FIG. 2A). At antibody concentrations of 165 ng/ml, 5A1 suppressed 50% of the observed hemolysis and 5C12 suppressed 90% of hemolysis observed in the hemolytic assay (FIG. 2A). 5A1 and 5C12 were further tested in a dose-response format; both 5A1 and 5C12 were shown to inhibit hemolysis in a dose-dependent manner (FIG. 2B).

These results demonstrate that not all C1s binding antibodies can neutralize complement mediated hemolysis. Two anti-C1s antibodies were identified, 5A1 and 5C12, that bind C1s and have substantial hemolysis neutralizing activity.

5A1 and 5C12 have also been shown to inhibit complement-dependent lysis of AQP4 expressing cells incubated with anti-AQP4 antibodies. This experiment demonstrated the activity of the anti-C1s antibodies 5A1 and 5C12 in a cellular model system of NMO and their utility for the treatment of NMO. See U.S. Provisional Application No. 61/810,222 and PCT App. No. PCT/US2014/33560.

### Example 4

### Anti-C1s Antibodies Inhibit C1s-Mediated Cleavage of C4

To analyze the ability of anti-C1s antibodies to neutralize the proteolytic activity of C1s, 5A1 and 5C12 were tested for their inhibitory activity on C1s-mediate cleavage of C4.

To this end, human C1s enzyme (2 ng; Complement Technology Inc., Catalog #A103) was incubated with an approximately 10-fold molar excess (25 ng) of C1s antibodies for 30 minutes at 4° C. Protein dilutions were made in PBS containing 0.1 mg gelatin/mL. The antibody/C1s mixture was incubated with 3 mg of human C4 protein (Complement Technology Inc. Cat # A105) for 5 minutes at 37° C. SDS-DTT Sample buffer was added to each sample, mixed and immediately placed in a 37° C. water bath for 15 min. The samples were loaded immediately onto a NuPage 10% BisTris SDS gel (Invitrogen Life Technologies) gel and ran for 1 hour at 150V. The gel was fixed for 1 hour, stained with Coomassie Blue for 24 hrs and de-stained overnight.

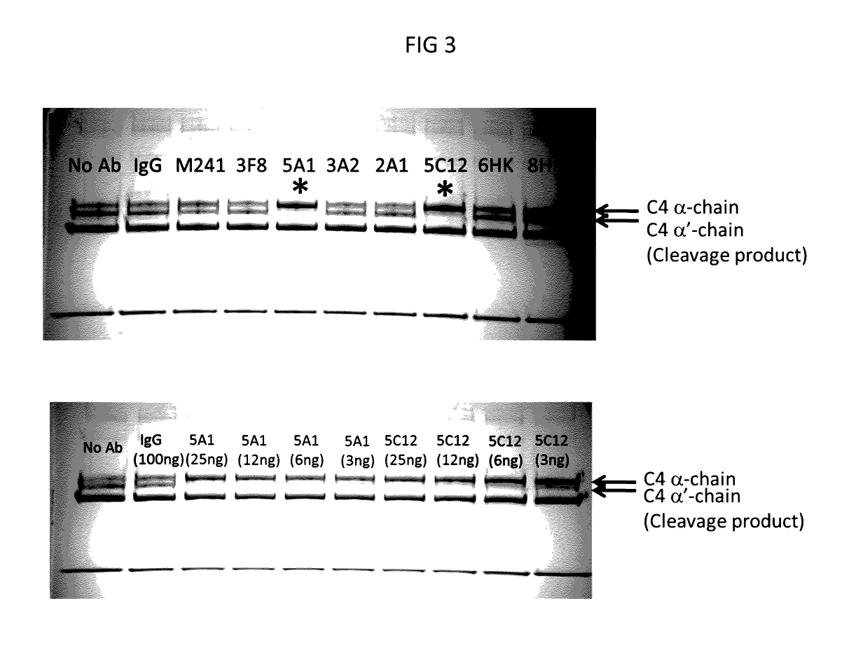

Eight anti-C1s antibodies were tested in the C4 cleavage assay. 5A1 and 5C12 inhibited C1s-mediated C4 α-chain cleavage if incubated at approximately 10-fold molar excess, whereas six other anti-C1s binding antibodies, including the C1s binding antibody 3F8 (see FIG. 1, Example 2), did not show inhibitory activity (FIG. 3—upper panel). Further testing in a dose-response format demonstrated that both 5A1 and 5C12 can inhibit C1s-mediated C4 cleavage at approximately equimolar concentrations of antibody (3 ng) and C1s (2 ng; FIG. 3—lower panel).

### Example 5

### Characterization of Binding Epitope Mapping for Antibody M1

### Anti-C1q Antibody M1 Kinetic Analyses

Antibody M1 binding data was compared with corresponding data obtained using the reference antibody 4A4B11. The 4A4B11 antibody is described in U.S. Pat. No. 4,595,654. The 4A4B11 producing hybridoma cell line is available from ATCC (ATCC HB-8327TM).

C1q-antibody interactions were measured using an OCTET™ System according to standard protocols and manufacturer's instructions. Briefly, human and mouse C1q proteins were immobilized separately on a biosensor at three concentrations (3 nM, 1.0 nM, and 0.33 nM). Next, the anti-C1q antibody M1 was injected onto the C1q-coated biosensor at a concentration of 2.0 μg/ml and the association constants (kₒₙ) and dissociation constants (k_{off}) for anti-C1q antibodies M1 and 4A4B11 were measured. The data were fit by non-linear regression analysis and using the Octet Data Analysis software to yield affinity (K_{D}) and kinetic parameters (k_{on/off}) for the interactions of M1 and 4A4B11 with human and mouse C1q respectively (Table 1).

TABLE 1
Kinetic Analysis of M1 and 4A4B11

| Antibody | Antigen | K_{D} (M) | kₒₙ (1/Ms) | k_{off} (1/s) |
|---|---|---|---|---|
| M1 | human C1q | 1.28*10⁻¹¹ | 5.18*10⁶ | 6.31*10⁻⁵ |
| M1 | mouse C1q | 3.23*10⁻¹¹ | 1.81*10⁶ | 5.84*10⁻⁵ |
| 4A4B11 | human C1q | 2.29*10⁻¹¹ | 4.49*10⁶ | 1.03*10⁻⁴ |
| 4A4B11 | mouse C1q | undetectable | undetectable | undetectable |

In this experimental series, anti-C1q antibody M1 was shown to bind both human and mouse C1q proteins with very high affinities (K_{D}<10⁻¹⁰ M). By comparison, the reference antibody 4A4B11 was found to bind to human C1q, whereas binding to mouse C1q was undetectable. Whereas the affinities of M1 and 4A4B11 for human C1q were on the same order of magnitude (i.e., in the double-digit picomolar range; K_{D}˜10-30 pM), the affinity of M1 for mouse C1q was determined to be about four orders of magnitude higher (K_{D}˜30 pM) than that the affinity of 4A4B11 for mouse C1q (K_{D}˜40 nM).

### Anti-C1q Antibodies M1 and 4A4B11 do not Compete for C1q-Binding

Blocking experiments were performed to determine whether the anti-C1q antibodies M1 and 4A4B11 bind to the same or overlapping epitopes of human C1q or whether M1 and 4A4B11 bind to separate C1q epitopes.

To this end, M1 was coated on a biosensor chip (BIACORE™) and subsequently contacted with a combination of human C1q and M1, a combination of human C1q and 4A4B11, or human C1q alone. C1q-binding to M1 was followed for 10 min and dissociation of M1-C1q complexes was subsequently followed for 20 min. Relative binding signals were recorded at the end of the association and dissociation periods. Table 2 shows the results of these experiments.

TABLE 2
Analysis of Simultaneous Interactions
of M1 and 4A4B11 with human C1q

| | | | Associa- | Dissocia- |
|---|---|---|---|---|
| Sensor | Antigen | Solution | tion Response | tion Response |
| Ab ID: | ID: | Ab ID: | (nm) @600 s | (nm) @1200 s |
| M1 | hC1q | M1 | −0.0119 | −0.00945 |
| M1 | hC1q | 4A4B11 | 0.8213 | 0.82139 |
| M1 | hC1q | None (Ag | 0.4715 | 0.45137 |
| | | Only) | | |

It was found that C1q alone bound effectively to immobilized M1 antibody on the biosensor chip. Preincubation of C1q with soluble M1 antibody prevented all binding of the resulting M1-C1q complex to immobilized M1. By contrast, preincubation of C1q with 4A4B11 did not prevent the interaction of the resulting 4A4B11-C1q complex with immobilized M1. The larger relative binding signals observed in the binding experiment involving the 4A4B11-C1q complex relative to the binding experiment involving C1q alone is due to the fact that the relative binding signals correlate with the molecular weight of the soluble binding partners and that the 4A4B11-C1q complex has a higher molecular weight than C1q alone.

These results demonstrate that 4A4B11 does not compete with M1 for C1q binding. Therefore, 4A4B11 and M1 recognize separate epitopes on C1q.

### Epitope Mapping

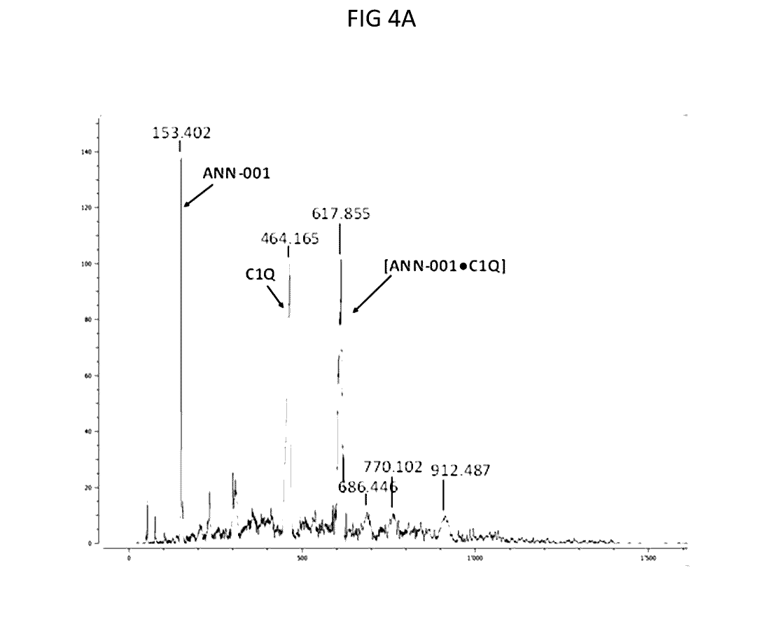

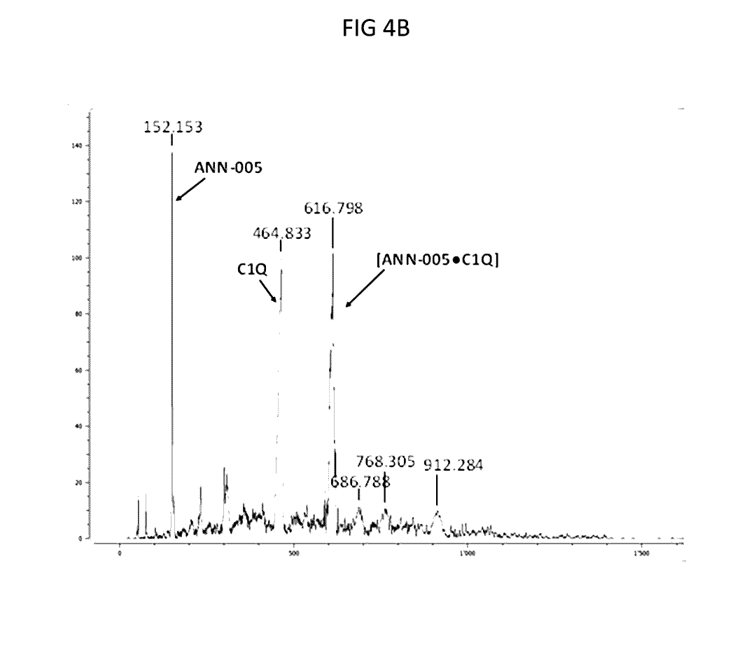

In order to determine the nature of the epitope (i.e. linear or conformational) bound by anti-C1q antibodies M1 and 4A4B11, the inhibition of the interaction between the C1Q protein and the antibodies 4A4B11 (ANN-001) and M1 (ANN-005) by unstructured peptides generated by proteolysis of the C1q antigen was evaluated. If the peptides generated by complete proteolysis of the antigen are able to inhibit the binding of the antigen on the antibody, the interaction is not based on conformation, and the epitope is linear. If the peptides generated by complete proteolysis of the antigen are unable to inhibit the binding of the antigen on the antibodies 4A4B11 and M1, the conformation is necessary for interaction. Based on the data described in detail below, unstructured peptides generated by digestion of native C1q did not compete with intact C1q for binding to the 4A4B11 (ANN-001) and M1 (ANN-005) antibodies (FIG. 4), suggesting that the C1q epitope for these antibodies is a complex conformational epitope.

In order to determine the key residues of the conformational C1q epitope that binds of ANN-001 and ANN-005 on C1Q antigen with high resolution the antibody/antigen complexes were incubated with deuterated cross-linkers and subjected to multi-enzymatic proteolytic cleavage. After enrichment of the cross-linked peptides, the samples were analyzed by high resolution mass spectrometry (nLC-Orbitrap MS) and the data generated analyzed using XQuest software. The analysis described below indicates that antibody 4A4B11 (ANN-001) binds to an epitope that includes amino acids 5202 and K219 of human C1QA and Y225 of human C1QC, and antibody M1 (ANN-005) binds to an epitope that includes amino acid K219 of human C1QA and S185 of human C1QC. See the amino acid sequence alignment of human and mouse C1qA and C1qC as shown below.

| Amino acid sequence alignment of human and mouse C1qA | |
|---|---|
| MEGPRGWLVLCVLAISLASMVTEDLCRAPDGKKGEAGRPGRRGRPGLKGEQGEPGAPGIR | human |
| METSQGWLVACVLTMTLVWTVAEDVCRAPNGKDGAPGNPGRPGRPGLKGERGEPGAAGIR | mouse |
| ** .:**** ***:::*. *:**:****:**.* .*.*** ********:*****.*** | |
| TGIQGLKGDQGEPGPSGNPGKVGYPGPSGPLGARGIPGIKGTKGSPGNIKDQPRPAFSAI | human |
| TGIRGFKGDPGESGPPGKPGNVGLPGPSGPLGDSGPQGLKGVKGNPGNIRDQPRPAFSAI | mouse |
| ***:*:*** **.**.*:**:** ******** * *:**.**.****:********** | |
| RRNPPMGGNVVIFDTVITNQEEPYQNHSGRFVCTVPGYYYFTFQVLSQWEICLSIVSSSR | human |
| RQNPMTLGNVVIFDKVLTNQESPYQNHTGRFICAVPGFYYFNFQVISKWDLCLFIKSSSG | mouse |
| *:** *********.*:****.*****:***:*:***:***.***:*:*::** * *** | |
| GQVRRSLGFCDTTNKGLFQVVSGGMVLQLQQGDQVWVEKDPKKGHIYQGSEADSVFSGFL | human |
| GQPRDSLSFSNTNNKGLFQVLAGGTVLQLRRGDEVWIEKDPAKGRIYQGTEADSIFSGFL | mouse |
| ** * **.*.:*.*******::** ****::**:**:**** **:****:****:***** | |
| IFPSA humanSEQ ID NO: 1 | |
| IFPSA mouseSEQ ID NO: 6 | |
| ***** | |
| Amino acid sequence alignment of human and mouse C1qC | |
| MDVGPSSLPHLGLKLLLLLLLLP-LRGQANTGCYGIPGMPGLPGAPGKDGYDGLPGPKGE | human |
| MVVGPSCQPPCGLCLLLLFLLALPLRSQASAGCYGIPGMPGMPGAPGKDGHDGLQGPKGE | mouse |
| * ****. ** ** ****:** **.**.:**********:********:*** ***** | |
| PGIPAIPGIRGPKGQKGEPGLPGHPGKNGPMGPPGMPGVPGPMGIPGEPGEEGRYKQKFQ | human |
| PGIPAVPGTRGPKGQKGEPGMPGHRGKNGPRGTSGLPGDPGPRGPPGEPGVEGRYKQKHQ | mouse |
| *****:** ***********:*** ***** * *:** *** * ***** ******* * | |
| SVFTVTRQTHQPPAPNSLIRFNAVLTNPQGDYDTSTGKFTCKVPGLYYFVYHASHTANLC | human |
| SVFTVTRQTTQYPEANALVRFNSVVTNPQGHYNPSTGKFTCEVPGLYYFVYYTSHTANLC | mouse |
| ********* * * .*:*:***:*:*****.*:.*******:*********::******* | |
| VLLYRSGVKVVTFCGHTSKTNQVNSGGVLLRLQVGEEVWLAVNDYYDMVGIQGSDSVFSG | human |
| VHLNLNLARVASFCDHMFNSKQVSSGGVLLRLQRGDEVWLSVNDYNGMVGIEGSNSVFSG | mouse |
| * * . .:*.:**.* :::**.********* *;****;**** .****:**:***** | |
| FLLFPD human SEQ ID NO: 3 | |
| FLLFPD mouse SEQ ID NO: 7 | |
| ****** | |

### 1. Identification of the C1q/Antibody Complexes by Mass Spectrometry

The C1q/antibody complexes were generated by mixing equimolar solutions of C1q antigen and antibody (4 μM in 5 μl each). One μl of the mixture obtained was mixed with 1 μl of a matrix composed of a re-crystallized sinapinic acid matrix (10 mg/ml) in acetonitrile/water (1:1, v/v), TFA 0.1% (K200 MALDI Kit). After mixing, 1 μl of each sample was spotted on the MALDI plate (SCOUT 384). After crystallization at room temperature, the plate was introduced in the MALDI mass spectrometer and analysed immediately. The analysis has been repeated in triplicate. FIG. 4 shows the presence of the antigen, antibody and antigen/antibody complexes for C1q/4A4B11 (FIG. 4A) and C1q/M1 (FIG. 4B). Peaks are present at the predicted molecular weights of monomeric antibody (˜150 kDa) and C1q monomer (˜460 kDa) and there is a 1:1 complex of antibody:antigen present at ˜615 kDa.

### 2. Unstructured C1q Peptides Generated by Proteolysis do not Compete for Binding of C1q to Antibody

To determine if the C1q/antibody complexes could be competed with peptides the C1q antigen was digested with immobilized pepsin. 25 μl of the antigen with a concentration of 10 μM were mixed with immobilized pepsin 5 μM and incubate at room temperature for 30 minutes. After the incubation time the sample was centrifuged and the supernatant was pipetted. The completion of the proteolysis was controlled by High-Mass MALDI mass spectrometry in linear mode. The pepsin proteolysis was optimized in order to obtain a large amount of peptide in the 1000-3500 Da range. Next, 5 μl of the antigen peptides generated by proteolysis were mixed with 5 μl of ANN-001 or ANN-005 (8 μM) and incubated at 37° for 6 hours. After incubation of ANN-001 or ANN-005 with the C1Q antigen peptides, 5 μl of the mixture was mixed with 5 μl of the C1Q antigen (4 μM) so the final mix contained 2 μM/2 μM/2.5 μM of C1Q antigen/4A4B11 or M1/C1Q antigen Peptides.

The MALDI ToF MS analysis was performed using CovalX's HM3 interaction module with a standard nitrogen laser and focusing on different mass ranges from 0 to 2000 kDa. For the analysis, the following parameters have been applied for Mass Spectrometer: Linear and Positive mode; Ion Source 1: 20 kV; Ion Source 2: 17 kV; Pulse Ion Extraction: 400 ns; for HM3: Gain Voltage: 3.14 kV; Gain Voltage: 3.14 kV; Acceleration Voltage: 20 kV.

To calibrate the instrument, an external calibration with clusters of Insulin, BSA and IgG has been applied. For each sample, 3 spots were analyzed (300 laser shots per spots). The presented spectrum corresponds to the sum of 300 laser shots. The MS data were analyzed using the Complex Tracker analysis software version 2.0 (CovalX Inc).

### 3. Identification of the Conformational Epitopes for C1q Binding to ANN-001 and ANN-005

Using chemical cross-linking, High-Mass MALDI mass spectrometry and nLC-Orbitrap mass spectrometry the interaction interface between the antigen C1Q and two monoclonal antibodies ANN-001 and ANN-005 was characterized. 5 μl of the sample C1Q antigen (concentration 4 μM) was mixed with 5 μl of the sample ANN-001 (Concentration 4 μM) or ANN-005 (Concentration 4 μM) in order to obtain an antibody/antigen mix with final concentration 2 μM/2 μM. The mixture was incubated at 37° C. for 180 minutes. In a first step, 1 mg of DiSuccinimidylSuberate H12 (DSS-H12) cross-linker was mixed with 1 mg of DiSuccinimidylSuberate D12 (DSS-D12) cross-linker. The 2 mg prepared were mixed with 1 ml of DMF in order to obtain a 2 mg/ml solution of DSS H12/D12. 10 μl of the antibody/antigen mix prepared previously were mixed with 1 μl of the solution of cross-linker d0/d12 prepared (2 mg/ml). The solution was incubated 180 minutes at room temperature in order to achieve the cross-linking reaction. In order to facilitate the proteolysis, it was necessary to reduce the disulfide bound present in this protein. The cross-linked sample was mixed with 20 μl of ammonium bicarbonate (25 mM, pH 8.3). After mixing 2.5 μl of DTT (500 mM) is added to the solution. The mixture was then incubated 1 hour at 55° C. After incubation, 2.5 μl of iodoacetamide (1M) was added before 1 hour of incubation at room temperature in a dark room. After incubation, the solution was diluted 1/5 by adding 120 μl of the buffer used for the proteolysis. 145 μl of the reduced/alkyled cross-linked sample was mixed with 2 μl of trypsin (Sigma, T6567). The proteolytic mixture was incubated overnight at 37° C. For α-chymotrypsin proteolysis, the buffer of proteolysis was Tris-HCL 100 mM, CaCl₂10 mM, pH7.8. The 145 μl of the reduced/alkyled cross-linked complex was mixed with 2 μl of α-chymotrypsin 200 μM and incubated overnight at 30° C. For this analysis, an nLC in combination with Orbitrap mass spectrometry were used. The cross-linker peptides were analyzed using Xquest version 2.0 and stavrox software. The peptides identified and cross-linked amino acids are indicated in Table 3 below.

TABLE 3
C1q cross-linked peptides and contact residues necessary for ANN-001 and
ANN-005 binding

| Protease | | | Contact | |
|---|---|---|---|---|
| Digest | X-linked Peptide | C1q Subunit | Residue | Antibody |
| Trypsin | GLFQVVSGGMVLQLQQGDQVWVE | C1qA | K219 | ANN-001 |
| | K(SEQ ID NO: 8) | | | |
| Trypsin | FQVVSGGMVLQL (SEQ ID NO: 9) | C1qA | 5202 | ANN-001 |
| Chymotrypsin | YDMVGIQGSDSVFSGF | C1qC | Y225 | ANN-001 |
| | (SEQ ID NO: 10 | | | |
| Trypsin | GLFQVVSGGMVLQLQQGDQVWVE | C1qA | K219 | ANN-005 |
| | K (SEQ ID NO: 11) | | | |
| Chymotrypsin | RSGVKVVTF (SEQ ID NO: 12) | C1qC | 5185 | ANN-005 |

### Example 6

### Materials and Methods

### Antibodies and Immunohistological Analysis

The IgM anti-GQ1b ganglioside mAb, CGM3 was derived from mice inoculated with a GT1a-bearing C. jejuni lipooligosaccharide (Goodyear et al., 1999). CGM3 reacts with gangliosides GQ1b, GD3, and GT1a that all share the terminal disialylgalactose structure. Previous in vitro studies have shown that CGM3 has similar ganglioside specificity and induces identical complement dependent pathogenic effects as human MFS sera (Goodyear et al., 1999; Plomp et al., 1999; Bullens et al., 2000; Jacobs et al., 2002; Jacobs et al., 2003). The control IgG1 antibody is a mouse monoclonal antibody obtained from Antibody Solutions (Cat #AP1-C). Normal human serum (NHS) was taken from a single donor stock that had been freshly frozen and stored in multiple aliquots at −70° C. to preserve complement activity. Prior to experimental use, CGM3 and NHS were dialysed for 24 h at 4° C. against Ringer's solution (116 mM NaCl, 4.5 mM KCl, 1 mM MgCl₂, 2 mM CaCl₂, 1 mM NaH₂PO₄, 23 mM NaHCO₃, 11 mM glucose, pH 7.4) and pre-gassed with 95% O₂/5% CO₂.

The intermediate complement component C3c was detected by incubation with FITC-labelled rabbit anti-C3c (1/300; Dako, Ely, UK); For neurofilament staining, sections of unfixed tissue were preincubated for 1 h at 4° C. with TRITC-conjugated a-BTx to label the nAChR at the NMJ, rinsed, immersed in ethanol at −20° C. for 20 min, then incubated overnight at room temperature with the rabbit polyclonal serum 1211 (1/750; reactive with phosphorylated neurofilament; Affiniti Research Products Ltd. Exeter, UK) followed by FITC conjugated goat anti-rabbit IgG (1/300; Southern Biotechnology Associates) for 3 h at 4° C. All detection antibodies were diluted in phosphate buffered saline (PBS).

### Ex Vivo Whole-Mount Muscle-Nerve Model of GBS

Mouse hemi-diaphragms with phrenic nerve attached (or triangularis sterni muscle in some cases for illustrative NMJ immunohistology) were dissected and mounted in Ringer's medium at room temperature (20-22° C.). Untreated small control sections were removed from each muscle preparation prior to any incubations and snap frozen on dry ice for subsequent baseline immunohistological analysis. Muscles were incubated with the anti-ganglioside monoclonal antibody CGM3 (50 μg/ml) for 2-2.5 h at 32° C., then for 30 min at 4° C. and then equilibrated for 10 min at room temperature, rinsed in Ringer's medium and subsequently exposed to 40% NHS in Ringer's medium for 1 h at room temperature. C1 antibodies (100 μg/ml) or the control mAb (100 μg/ml) was mixed with NHS 10 min prior to the incubation of the muscle preparation. The intermediate complement component C3c was detected by incubation with FITC-labelled rabbit anti-C3c (1/300; Dako, Ely, UK) for 1 h at 4° C. Digital images were captured using both a Zeiss Pascal confocal laser scanning microscope and a Zeiss Axio Imager Z1 with ApoTome. Image-analysis measurements were made using ImageJ (NIH, USA) image analysis software. For quantitative analysis of C3c, MAC and Nfil three staining runs of each marker were performed on tissue from at least three individual hemi-diaphragms, and quantified as previously described (O'Hanlon et al., 2001).

### In Vivo Model of GBS

Balb/c mice (3-4 weeks old, 10-15 g) were injected intraperitoneally with 1.5 mg CGM3, followed 16 h later by an intravascular (IV) injection of 200 mg of anti-C1q (i.e., M1) antibody and intraperitoneal (IP) injection of 0.5 ml 100% NHS. After 4 hours the mice were euthanized with CO₂ and diaphragm muscle tissue was dissected and processed for immunohistological analyses (see Examples 7-9 below). The in vivo effect of the anti-C1q antibodies 4A4B11 and M1 on diaphragm C3c deposition and axonal integrity was determined.

### Production and Characterization of Anti-C1q and Anti-C1s Antibodies

The anti-C1q antibody 4A4B11 is described in U.S. Pat. No. 4,595,654 and the hybridoma for this line is available from ATCC (ATCC HB-8327TM). The anti-C1q antibody M1 was generated by Antibody Solutions Inc. (Sunnyvale Calif.) by immunizing C1q knockout mice with human C1q using standard mouse immunization and hybridoma screening technologies (Milstein, C (1999). Bioessays 21: 966-73; Mark Page, Robin Thorpe, The Protein Protocols Handbook 2002, Editors: John M. Walker, pp 1111-1113). The generation and characterization of this antibody is described in US patent application (Ser. No. 61/844,368).

The anti-C1s antibody 5C12 was generated and characterized as described in Example 1 above.

### Example 7

### C1 Antibodies Prevent C3c Deposition in an Ex Vivo Whole-Mount Muscle Model of GBS

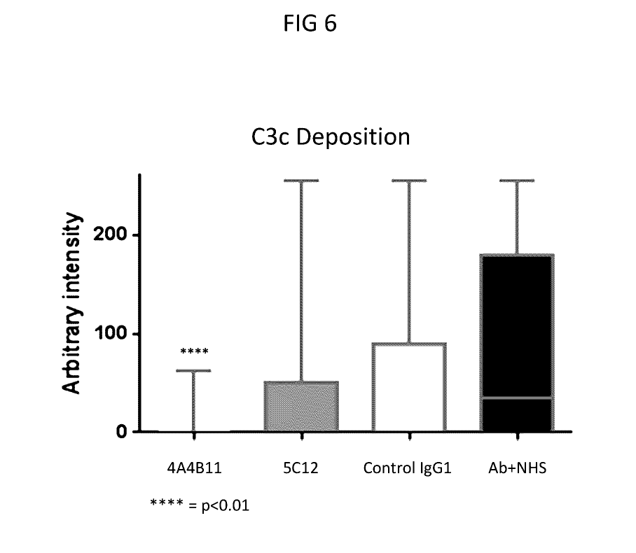

Anti-C1q and anti-C1s antibody efficacy was tested in an ex vivo diaphragm model of GBS in which muscles with phrenic nerve attached were incubated with the anti-ganglioside monoclonal antibody CGM3 (50 mg/ml) for 2-2.5 h at 32° C., then for 30 min at 4° C., and then equilibrated for 10 min at room temperature, rinsed in Ringer's medium, and subsequently exposed to 40% NHS in Ringer's medium for 1 h at room temperature. C1 antibodies (100 μg/ml) or the control mAb (100 μg/ml) was mixed with NHS 10 min prior to the incubation of the muscle preparation. An anti-ganglioside monoclonal antibody and human complement (i.e., Ab+NHS) causes complement deposition and damage of motor neuron axons. FIG. 6 shows that incubation of the anti-C1q mAb with the Ab+NHS causes a statistically significant reduction in C3c deposition relative to all of the other groups. The 5C12 antibody shows a reduction in C3c deposition but it does not appear to be statistically significant in this experiment.

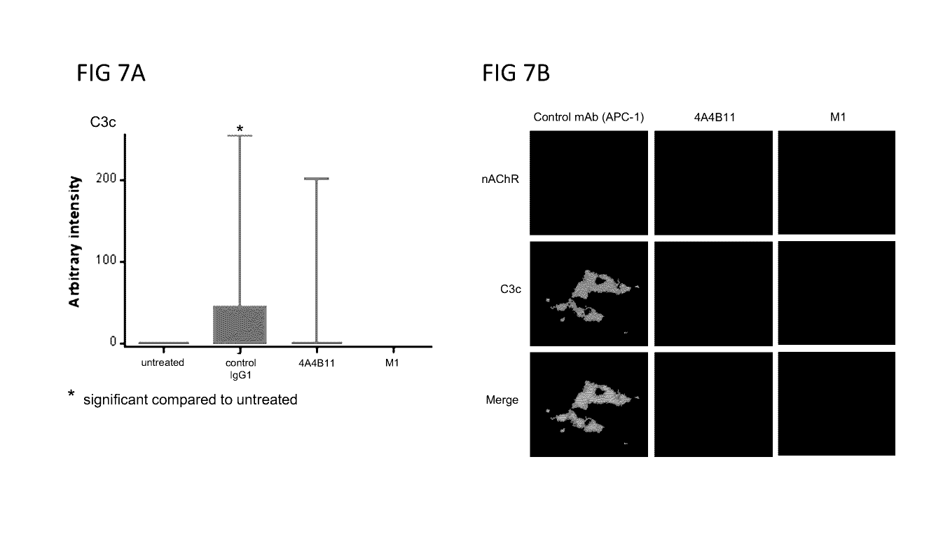

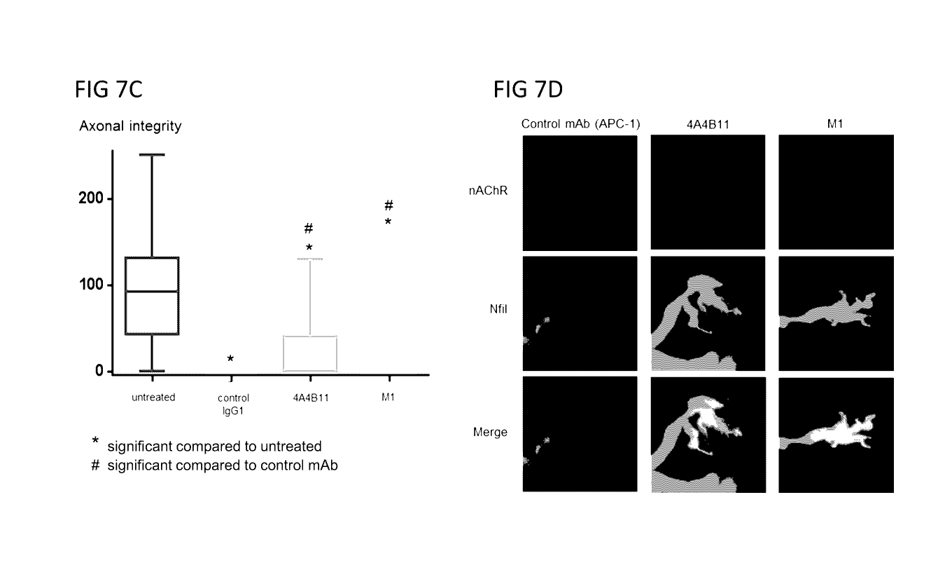

It was also demonstrated that anti-C1q antibodies 4A4B11 and M1 can suppress complement deposition and preserve axonal integrity in the ex-vivo diaphragm model of GBS. FIG. 7A shows a box and whisker plot of the quantitation of the immunofluorescent labeling of C3c deposition on the explanted diaphragm. The control antibody showed a statistically significant increase in C3c staining compared to the untreated tissue (*−p<0.01; Mann-Whitney test), while the anti-C1q antibodies were not different than untreated tissue (FIG. 7A). FIG. 7B shows images of sections quantitated in FIG. 7A. The tissues were either untreated (no complement added) or treated with a control IgG1 antibody or the anti-C1q antibodies 4A4B11 and M1. The results demonstrate that addition of anti-C1q antibodies resulted in almost complete elimination of C3c deposition at the NMJ (FIG. 7B), compared to tissue treated with the control antibody (FIG. 7B, left column). FIG. 7C shows quantitation of the neurofilament staining at the NMJ, demonstrating that anti-C1q antibody treatment causes a statistically significant increase, compared to the control antibody, in neurofilament staining representative of increased axonal integrity. FIG. 7D shows representative images of NMJ showing the post-synaptic membrane (nAChR) and axon (Nfil) staining in the presence of the control antibody and the anti-C1q antibodies. The images demonstrate that the diminished axonal neurofilament labeling in samples treated with the control antibody treated samples is significantly restored by treatment with the anti-C1q antibodies (FIG. 7D). Both the anti-C1q antibody M1 and the anti-C1q antibody 4A4B11 caused a statistically significant increase in neurofilament staining (i.e., axonal integrity) relative to the control IgG1 antibody (*/#—p<0.01; Mann-Whitney test) (FIG. 7C).

### Example 8

### C1q Antibody M1 Prevents Axonal Damage Formation in an In Vivo Mouse Model of GBS

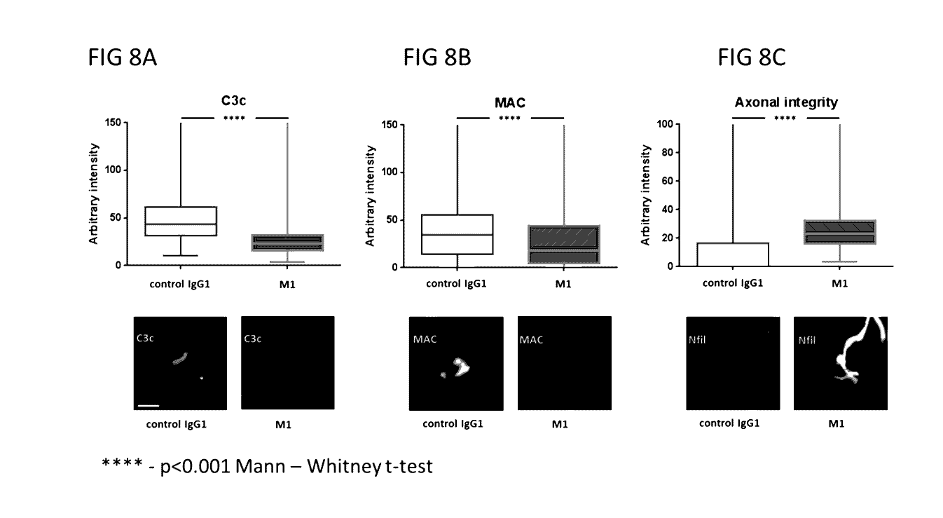

The efficacy of the anti-C1q antibody M1 was tested in an in vivo mouse model of GBS in which 1.5 mg anti-ganglioside antibody (n=7) was injected by IP and 16 h later, IV injection of 200 mg M1 (˜15 mg/kg) (n=4) or IgG1 control antibody (Antibody Solutions AP1-C) (n=3) followed by IP administration of 100% normal human serum. After 4 hours diaphragm tissue was collected and processed for C3c deposition, and axonal integrity over the endplate was assessed by neurofilament staining with a polyclonal rabbit antibody to phosphorylated neurofilament. Mann Whitney test was used to assess the statistical significance of the results (***=p<0.001). FIG. 8A shows a box and whisker plot of the quantitation of the C3c immunofluorescence at the motor nerve endplate and below the graph is an image of C3c (green) deposition at the NMJ with muscle fluorescently labeled by α-bungarotoxin (BTx; red). This image demonstrates the inhibition of C3c deposition by the M1 antibody. FIG. 8B shows a box and whisker plot of the quantitation of the MAC immunofluorescence at the motor nerve endplate and a corresponding image of MAC (green) deposition at the NMJ with muscle fluorescently labeled by BTx (red) demonstrating that M1 treatment causes blockade of MAC deposition and thus blockade of complement factors upstream (C3c) and downstream (MAC) of the C5 protein, which is the target of Eculizumab function. FIG. 8C shows quantitation of the neurofilament staining at the NMJ and an image of the neorofilament staining demonstrating that M1 treatment causes a dramatic increase in neurofilament staining representative of increased axonal integrity.

### Example 9

### C1q Antibody M1 Decreases Respiratory Muscle Damage in an In Vivo Mouse Model of GBS

The efficacy of the anti-C1q antibody M1 in treating respiratory muscle damage was tested in an in vivo mouse model of GBS.

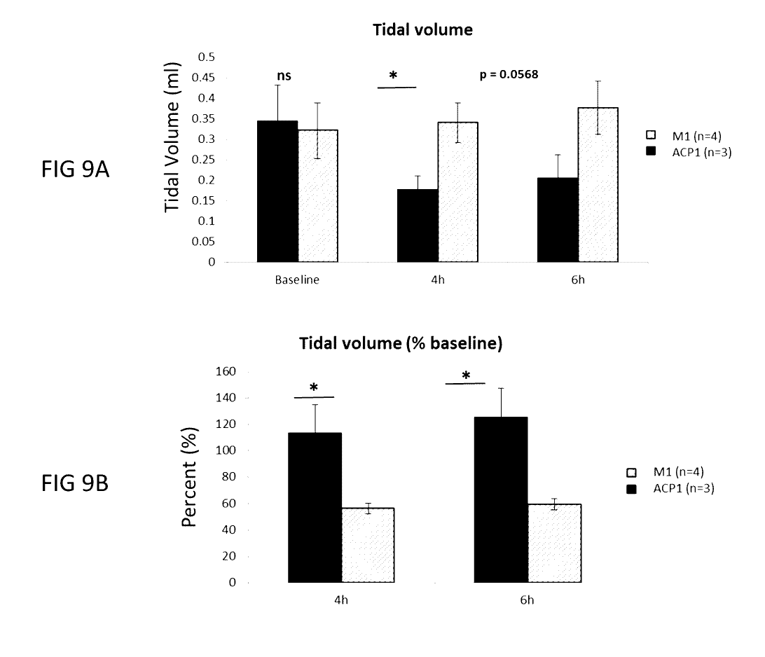

In this in vivo mouse model of GBS, it is a part of the autoimmune disease process that attacks the motor nerve terminals at the diaphragm and blocks normal nerve transmission to the diaphragm. Injury was induced in seven mice by injecting the mice by intraperitoneal (IP) injection with 1.5 mg anti-ganglioside antibody combined with serum. The following day mice were injected by IV injection with 50 mg/kg of anti-C1q antibody M1 (n=4) or isotype control antibody ACP1 (Antibody Solutions, Mountain View, Calif.) (n=3), followed by IP injection of normal human serum (NHS) 30 min later. Baseline tidal volume readings were recorded the day before antibody injection and subsequent readings were taken at 4 h and 6 h post-injury induction (FIG. 9A).

FIG. 9B shows the percentage change from baseline tidal volume for the experimental groups. The results depicted in FIG. 9 demonstrate that tidal volume is significantly reduced, compared to baseline volume, in mice treated with the isotype control ACP-1 antibody. However, normal tidal volumes were maintained in mice treated with the M1 antibody.

### Example 10

### Test for C1q Antibody as a Therapeutic Agent in a GBS Clinical Trial

The efficacy of a humanized C1q antibody is tested in a clinical trial for GBS as previously described (e.g., Clinical Trials website for GBS study with Eculizumab).

Briefly, the incidence of adverse effects and severe adverse is first determined with the humanized anti-C1q antibody and IVIg compared to placebo controls over a 6-18 month period following intravenous delivery. Improvement of one or more grade in functional outcome (on the 6 point GBS disability scale) is then monitor at 4 weeks. The ability to walk unaided (GBS disability score 2) is also monitored at 8 weeks. The following effects are also monitored: time taken to improve by at least one grade (on the GBS disability scale) at 8 weeks, the time taken to walk independently at 1 year, difference in GBS disability score at maximum disability completed with 6 months, percentage of patients with a clinically relevant improvement in R-ODS score at 6 months, increase from baseline in R-ODS score by at least 6 points on the centile metric score at 4 weeks and 6 months, percentage of patients with a clinically relevant improvement in ONLS at 6 months (defined as an increase from baseline in ONLS score by at least 1 point at 4 weeks and 6 months), requirement for ventilatory support (GBS disability score 5) over 4 weeks, duration of ventilatory support over 8 weeks, occurrence of relapse over 2 years, and mortality within the first 6 months.

Eligibility criteria include, without limitation: age of 18 years and older; both genders; patients aged >18 years diagnosed with GBS according to NINDS diagnostic criteria; onset of weakness due to GBS is less than 2 weeks ago; patients who are unable to walk unaided for >10 meters (grade >3 on GBS disability scale); patients who are being considered for or already on IVIg treatment. The first dose of anti-C1q antibody treatment may be started within 2 weeks from onset of weakness and any time during the IVIg treatment period. Signed informed consent is also obtained.

Exclusion criteria include, without limitation: age <18 years; patients who are pregnant or lactating; patients that show clear clinical evidence of a polyneuropathy caused by e.g., diabetes mellitus (except mild sensory), alcoholism, severe vitamin deficiency, and porphyria; patients having received immunosuppressive treatment (e.g., azathioprine, cyclosporine, mycofenolatemofetil, tacrolimus, sirolimus or >20 mg prednisolone daily) during the previous month; patients known to have severe concurrent disease, like malignancy, severe cardiovascular disease, AIDS, severe COPD, TB, etc.; inability to comply with study related procedures or appointments during 6 months; any condition that in the opinion of the investigator could increase the patient's risk by participating in the study or confound the outcome of the study; unresoled Neisseria meningitidis infection or history of meningococcal infection; patients unsuitable for antibiotic prophylaxis (e.g., due to allergy); known hypersensitivity to anti-C1q antibody, murine proteins, or to any of the excipients; patients known or suspected of hereditary complement deficiencies, and women of child-bearing potential who are unwilling to use effective contraception during treatment and for 5 months after treatment is completed.

### Deposit of Material

The following materials have been deposited according to the Budapest Treaty in the American Type Culture Collection, ATCC Patent Depository, 10801 University Blvd., Manassas, Va. 20110-2209, USA (ATCC):

| | | Deposit | ATCC |
|---|---|---|---|
| Sample ID | Isotype | Date | Accession No. |
| Mouse anti-C1s-RP mAB cell line | IgG1, | May 15, | PTA-120351 |
| 5A1 IgG1 producing antibody 5A1 | kappa | 2013 | |
| Mouse anti-C1s-RP mAb cell line | IgG1, | May 15, | PTA-120352 |
| 5C12 IgG1 producing antibody 5C12 | kappa | 2013 | |
| Mouse hybridoma C1qM1 7788-1(M) | IgG1, | Jun. 6, | PTA-120399 |
| 051613 producing anti-C1q antibody | kappa | 2013 | |
| M1 | | | |

The hybridoma cell lines producing the 5A1 antibody (mouse anti-C1s-RP mAb cell line 5A1 IgG1), the 5C12 antibody (mouse anti-C1s-RP mAb cell line 5C12 IgG1), and M1 antibody (mouse hybridoma C1qM1 7788-1(M) 051613) have each been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. Each of the deposits is available as required by foreign patent laws in countries wherein counterparts of the subject application, or its progeny are filed. However, it should be understood that the availability of the deposits does not constitute a license to practice the subject invention in derogation of patent rights granted by governmental action.

### REFERENCES

Aberer, W, Ann Med. 2012, 44(6), 523-9.
Ayzenberg, I et al., JAMA Neurol. 2013, 1-4.
Bennett, J L, et al., Ann Neurol. 2009, 66(5), 617-629.
Botto, M et al., Nature Genetics, 1998, 19, 56-59.
Botto, M, et al., J Biol Chem. 2011, 286(18), 16516-16524.
Bullens R W et al., Muscle Nerve. 2000 July; 23(7):1035-43.
Burt, C C. and Arrowsmith, J E. (2009). Surgery (Oxford) 27 (11): 475-479
Gadjeva, M G et al., Allergy Asthma Proc. 2012, 33, Suppl 1:S73-6.
Ghai, R, et al., Immunobiology 2007, 212(4-5), 253-66.
Gomez, A M et al., Autoimmunity 2010, 43, 353-370.
Gómez-Almaguer, D, Hematology. 2012, Suppl 1:S25-7.
Goodyear C S et al., J Clin Invest. 1999 September; 104(6):697-708.
Greenberg, B M et al., Mult Scler. 2012, 18(7), 1022-6.
Hafer-Macko C et al., Ann Neurol. 1996 October; 40(4):635-44.
Halstead S K et al., Brain. 2008 May; 131(Pt 5):1197-208.
Hartung H P et al., Neurology. 1987 June; 37(6):1006-9.
Hengstman, G J et al., Mult Scler. 2007, 13(5), 679-682.
Hinson, S R et al., Neurology. 2007, 69(24), 2221-31.
Hinson, S R et al., Arch. Neurol. 2009, 66, 1164-1167.
Ho T W et al., Ann Neurol 45:168-173.
Hughes R A et al., Neurology 61 (6): 736-40
Hughes R A and Cornblath D R, Lancet. 2005 Nov. 5; 366(9497):1653-66.
Jacobs B C et al., Muscle Nerve. 2002 April; 25(4):549-58.
Jacobs B C et al., Brain. 2003 October; 126(Pt 10):2220-34.
Jacob, A et al., J Neurol Neurosurg Psychiatry. 2012, epub.
Jujus, S et al., Nat Clin Pract Neurol. 2008, 4(4), 202-214.
Jujus, S, and Wildemann, B, Nat Rev Neurol. 2010, 6(7), 383-392.
Kieseier, B C et al., Arch Neurol. 2012, 24, 1-4.
Kim, S H et al., J Clin Neurol. 2013, 9(1), 36-42.
Kira, J, J Neurol Sci. 2011, 311(1-2), 69-77.
Kishore, U, and Reid, K B M, Immunopharmacology 2000, 49, 159.170.
Klos, A et al., Mol Immunol. 2009, 46(14), 2753-66.
Koski C L et al., J Clin Invest. 1987 November; 80(5):1492-7.
Lennon, V A et al., J Exp Med. 2005, 202(4), 473-477.
Lucchinetti, C F et al., Brain. 2002, 125(Pt 7), 1450-1461.
Manderson, A P et al., Annu. Rev. Immunol. 2004, 22, 431-456.
Misu, T et al., Brain. 2007, 130(Pt 5), 1224-1234.
Nayak, A et al., Innate Immun. 2012, 18(2), 350-63.
O'Hanlon G M et al., Brain. 2001 May; 124(Pt 5):893-906.
Papadopoulos, M C and Verkman, A S, Lancet Neurol. 2012, 11(6), 535-544.
Papadopoulos, M. C. and A. S. Verkman, Nature Rev. Neurosci. 2013, in press.
Parker, C, Lancet. 2009; 373(9665), 759-767.
Phuan, P W et al., J Biol Chem. 2012, 287(17), 13829-13839.
Ratelade, J et al., Acta Neuropathol. 2012, 123(6), 861-872.
Ricklin, D and Lambris, J D, Adv Exp Med Biol. 2013, 735, 1-22.
Ricklin, D et al., Nat Immunology. 2010, 11(9), 785-797.
Risitano, A M, Adv Exp Med Biol. 2013, 735, 155-72.
Roemer, S F et al., Brain. 2007, 130 (Pt 5), 1194-1205.
Rossi A et al., Glia. 2012, 60(12), 2027-2039.
Saadoun, S et al., Brain. 2010, 133(Pt 2), 349-361.
Saadoun, S et al., Ann Neurol. 2012, 71(3), 323-333.
Sabater, L et al., J. Neuroimmunol. 2009, 215, 31-35.
Sanders M E et al., J Immunol. 1986 Jun. 15; 136(12):4456-9
Sarzi-Puttini, P et al., Autoimmunity. 2005, 38(7), 507-18.
Sato, D et al., Arq Neuropsiquiatr. 2012, 70(1), 59-66.
Susuki K et al., J Neurol Sci. 2003 Jul. 15; 211(1-2):89-92.
Susuki K et al., J Neurosci. 2007 Apr. 11; 27(15):3956-67.
Tradtrantip, L et al., FASEB J. 2012a, 26, 2197-2208.
Tradtrantip, L et al., Ann. Neurol. 2012b, 71, 314-322.
Tradtrantip, L et al., Ann Neurol. 2013, 73(1), 77-85.
Tüzün, E et al., Adv Exp Med Biol. 2008, 632, 265-72.
Van Doom P A et al., Lancet Neurol. 2008 October; 7(10):939-50.
Van Herle et al., Annu Rev Pharmacol Toxicol. 2012, 52, 523-47.
Vedove, C D et al., Arch Dermatol Res. 2009, 301(1), 99-105.
Wallis, R et al., Immunobiol. 2010, 215, 1-11.
Wilde, B et al., Kidney Int. 2011, 79, 599-612.
Willison H J et al., Brain. 2002 December; 125(Pt 12):2591-625.
Wingerchuk, D M, J Clin Immunol. 2013, 33, Suppl 1:S33-7.
Wingerchuk, D M et al., Lancet Neurol. 2007, 6(9), 805-815.
Yuki N et al., Ann Neurol. 2001 June; 49(6):712-20.
Zhang, H et al., Ann Neurol. 2011, 70(6), 943-954.
Zhang, H and Verkman A S, J. Clin. Invest. 2013, in press
Zipfel, P F and Skerka, C, 2008, eLS.
Zipfel, P F and Skerka, C, Nature Rev. Immunol. 2009, 9, 729-740.

1. A method of treating or preventing Guillain-Barré Syndrome (GBS), comprising administering to an individual an antibody, wherein the antibody is:
a) an anti-C1q antibody, wherein the anti-C1q antibody inhibits the interaction between C1q and an autoantibody, or between C1q and C1r, or between C1q and C1s, or wherein the anti-C1q antibody prevents C1q from activating C1r or C1s;
b) an anti-C1s antibody, wherein the anti-C1s antibody inhibits the interaction between C1s and C1q, or between C1s and C1r, or between C1s and C2, or between C1s and C4, or wherein the anti-C1s antibody inhibits the catalytic activity of C1s or inhibits the processing of pro-C1s to an active protease;
c) an anti-C1r antibody, wherein the anti-C1r antibody inhibits the interaction between C1r and C1q, or between C1r and C1s, or wherein the anti-C1r antibody inhibits the catalytic activity of C1r or inhibits the processing of pro-C1r to an active protease; or
d) an anti-C1 complex antibody that binds to a combinatorial epitope within the C1 complex, wherein said combinatorial epitope is comprised of C1q and C1s; C1q and C1r; C1r and C1s; or C1q, C1r, and C1s; or wherein the anti-C1 complex antibody inhibits C1r or C1s activation or prevents their ability to act on C2 or C4.

2. The method of claim 1, wherein the antibody binds C1q, C1r, or C1s.

3. The method of claim 1, wherein the antibody is an anti-C1q antibody.

4. The method of claim 3, wherein the anti-C1q antibody is:
a) an anti-C1q antibody comprising a light chain variable domain and a heavy chain variable domain, wherein the light chain variable domain comprises the HVR-L1, HVR-L2, and HVR-L3 of the monoclonal antibody M1 produced by a hybridoma cell line with ATCC Accession Number PTA-120399 or progeny thereof; and/or wherein the heavy chain variable domain comprises the HVR-H1, HVR-H2, and HVR-H3 of the monoclonal antibody M1 produced by a hybridoma cell line with ATCC Accession Number PTA-120399 or progeny thereof; or
b) an anti-C1q antibody which binds essentially the same C1q epitope as the antibody M1 produced by the hybridoma cell line with ATCC Accession Number PTA-120399 or anti-C1q binding fragments thereof.

5. The method of claim 3, wherein the anti-C1q antibody is an anti-C1q antibody that binds to a C1q protein and binds to one or more amino acids of the C1q protein within amino acid residues selected from:
a) amino acid residues 196-226 of SEQ ID NO:1, or amino acid residues of a C1 q protein chain A (C1qA) corresponding to amino acid residues 196-226 (GLFQVVSGGMVLQLQQGDQVWVEKDPKKGHI) of SEQ ID NO:1;
b) amino acid residues 196-221 of SEQ ID NO:1, or amino acid residues of a C1qA corresponding to amino acid residues 196-221 (GLFQVVSGGMVLQLQQGDQVWVEKDP) of SEQ ID. NO:1;
c) amino acid residues 202-221 of SEQ ID NO:1, or amino acid residues of a C1qA corresponding to amino acid residues 202-221 (SGGMVLQLQQGDQVWVEKDP) of SEQ ID NO:1;
d) amino acid residues 202-219 of SEQ ID NO:1, or amino acid residues of a C1qA corresponding to amino acid residues 202-219 (SGGMVLQLQQGDQVWVEK) of SEQ ID NO:1;
e) amino acid residues Lys 219 and/or Ser 202 of SEQ ID NO:1, or amino acid residues of a C1qA corresponding Lys 219 and/or Ser 202 of SEQ ID NO:1;
f) amino acid residues 218-240 of SEQ ID NO:3 or amino acid residues of a C1q protein chain C (C1qC) corresponding to amino acid residues 218-240 (WLAVNDYYDMVGIQGSDSVFSGF) of SEQ ID NO:3;
g) amino acid residues 225-240 of SEQ ID NO:3 or amino acid residues of a C1qC corresponding to amino acid residues 225-240 (YDMVGI QGSDSVFSGF) of SEQ ID NO:3;
h) amino acid residues 225-232 of SEQ ID NO:3 or amino acid residues of a C1qC corresponding to amino acid residues 225-232 (YDMVGIQG) of SEQ ID NO:3;
i) amino acid residue Tyr 225 of SEQ ID NO:3 or an amino acid residue of a C1qC corresponding to amino acid residue Tyr 225 of SEQ ID NO:3;
j) amino acid residues 174-196 of SEQ ID NO:3 or amino acid residues of a C1qC corresponding to amino acid residues 174-196 (HTANLCVLLYRSGVKVVTFCGHT) of SEQ ID NO:3;
k) amino acid residues 184-192 of SEQ ID NO:3 or amino acid residues of a C1qC corresponding to amino acid residues 184-192 (RSGVKVVTF) of SEQ ID NO:3;
l) amino acid residues 185-187 of SEQ ID NO:3 or amino acid residues of a C1qC corresponding to amino acid residues 185-187 (SGV) of SEQ ID NO:3; and
m) amino acid residue Ser 185 of SEQ ID NO:3 or an amino acid residue of a C1qC corresponding to amino acid residue Ser 185 of SEQ ID NO:3.

6. (canceled)

7. The method of claim 3, wherein the anti-C1q antibody is an anti-C1q antibody that binds to a C1q protein and binds to one or more amino acids of the C1q protein chain A (C1qA) within amino acid residues selected from:
a) amino acid residues 196-226 of SEQ ID NO:1, or amino acid residues of a C1q protein chain A (C1qA) corresponding to amino acid residues 196-226 (GLFQVVSGGMVLQLQQGDQVWVEKDPKKGHI) of SEQ ID NO:1;
b) amino acid residues 196-221 of SEQ ID NO:1, or amino acid residues of a C1qA corresponding to amino acid residues 196-221 (GLFQVVSGGMVLQLQQGDQVWVEKDP) of SEQ ID. NO:1;
c) amino acid residues 202-221 of SEQ ID NO:1, or amino acid residues of a C1qA corresponding to amino acid residues 202-221 (SGGMVLQLQQGDQVWVEKDP) of SEQ ID NO:1;
d) amino acid residues 202-219 of SEQ ID NO:1, or amino acid residues of a C1qA corresponding to amino acid residues 202-219 (SGGMVLQLQQGDQVWVEK) of SEQ ID NO:1; and
e) amino acid residue Lys 219 of SEQ ID NO:1, or an amino acid residue of a C1qA corresponding Lys 219 of SEQ ID NO:1; and
wherein the anti-C1q antibody binds to one or more amino acids of the C1q protein chain C (C1qC) within amino acid residues selected from:
f) amino acid residues 174-196 of SEQ ID NO:3 or amino acid residues of a C1qC corresponding to amino acid residues 174-196 (HTANLCVLLYRSGVKVVTFCGHT) of SEQ ID NO:3;
g) amino acid residues 184-192 of SEQ ID NO:3 or amino acid residues of a C1qC corresponding to amino acid residues 184-192 (RSGVKVVTF) of SEQ ID NO:3;
h) amino acid residues 185-187 of SEQ ID NO:3 or amino acid residues of a C1qC corresponding to amino acid residues 185-187 (SGV) of SEQ ID NO:3; and
i) amino acid residue Ser 185 of SEQ ID NO:3 or an amino acid residue of a C1qC corresponding to amino acid residue Ser 185 of SEQ ID NO:3.

8. The method of claim 3, wherein the anti-C1q antibody binds specifically to both human C1q and mouse C1q.

9. The method of claim 3, wherein the anti-C1q antibody has dissociation constant (K_{D}) for human C1q and mouse C1q of less than 100 pM.

10. The method of claim 3, wherein the anti-C1q antibody specifically binds to and inhibits a biological activity of C1q.

11. The method claim 10, wherein the biological activity is (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to beta-amyloid, (10) C1q binding to calreticulin, (11) activation of the classical complement activation pathway, (12) activation of antibody and complement dependent cytotoxicity, (13) CH50 hemolysis, (14) synapse loss, (15) B-cell antibody production, (16) dendritic cell maturation, (17) T-cell proliferation, (18) cytokine production (19) microglia activation, (20) Arthus reaction, (21) phagocytosis of synapses or nerve endings, or (22) activation of complement receptor 3 (CR3/C3) expressing cells.

12. (canceled)

13. The method of claim 1, wherein the antibody is an anti-C1s antibody.

14. The method of claim 13, wherein the anti-C1s antibody is:
a) an anti-C1s antibody comprising a light chain variable domain and a heavy chain variable domain, wherein the light chain variable domain comprises the HVR-L1, HVR-L2, and HVR-L3 of the monoclonal antibody 5A1 produced by a hybridoma cell line with ATCC Accession Number PTA-120351, or progeny thereof;
b) an anti-C1s antibody comprising a light chain variable domain and a heavy chain variable domain, wherein the heavy chain variable domain comprises the HVR-H1, HVR-H2, and HVR-H3 of the monoclonal antibody 5A1 produced by a hybridoma cell line with ATCC Accession Number PTA-120351, or progeny thereof;
c) an anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5A1 produced by a hybridoma cell line with ATCC Accession Number PTA-120351 or progeny thereof;
d) an anti-C1s antibody comprising a light chain variable domain and a heavy chain variable domain, wherein the light chain variable domain comprises the HVR-L1, HVR-L2, and HVR-L3 of the monoclonal antibody 5C12 produced by a hybridoma cell line with ATCC Accession Number PTA-120352, or progeny thereof;
e) an anti-C1s antibody comprising a light chain variable domain and a heavy chain variable domain, wherein the heavy chain variable domain comprises the HVR-H1, HVR-H2, and HVR-H3 of the monoclonal antibody 5C12 produced by a hybridoma cell line with ATCC Accession Number PTA-120352, or progeny thereof;
f) an anti-C1s antibody comprising a light chain variable domain and a heavy chain variable domain, wherein the light chain variable domain comprises the HVR-L1, HVR-L2, and HVR-L3 and the heavy chain variable domain comprises the HVR-H1, HVR-H2, and HVR-H3 of the monoclonal antibody 5C12 produced by a hybridoma cell line with ATCC Accession Number PTA-120352, or progeny thereof;
g) an murine anti-human C1s monoclonal antibody 5A1 produced by a hybridoma cell line with ATCC Accession Number PTA-120351, or progeny thereof;
h) an murine anti-human C1s monoclonal antibody 5C12 produced by a hybridoma cell line with ATCC Accession Number PTA-120352 or progeny thereof;
i) an anti-C1s antibody which binds essentially the same C1s epitope as the antibody 5A1 produced by a hybridoma cell line with ATCC Accession Number PTA-120351; or
j) an anti-C1s antibody which binds essentially the same C1s epitope as the 5C12 antibody produced by a hybridoma cell line with ATCC Accession Number PTA-120352.

15. The method of claim 14, wherein the anti-C1s antibody specifically binds to and inhibits a biological activity of C1s or the C1s proenzyme.

16. The method of claim 15, wherein the biological activity is (1) C1s binding to C1q, (2) C1s binding to C1r, (3) C1s binding to C2 or C4, (4) the proteolytic enzyme activity of C1s, (5) the conversion of the C1s proenzyme to an active protease, or (6) cleavage of C4, (7) activation of the classical complement activation pathway, (8) activation of antibody and complement dependent cytotoxicity, or (9) C1F hemolysis.

17-20. (canceled)

21. The method of claim 1, wherein the antibody is an anti-C1r antibody.

22. The method of claim 1, wherein the antibody is an anti-C1 complex antibody.

23. The method of claim 1, wherein the antibody binds human C1q, C1r, or C1s, or wherein the antibody binds human C1 complex.

24. (canceled)

25. The method of claim 1, wherein the antibody is a monoclonal antibody.

26. The method of claim 1, wherein the antibody is a mouse antibody, a human antibody, a humanized antibody, or a chimeric antibody.

27. The method claim 1, wherein the antibody is an antibody fragment selected from Fab, Fab′-SH, Fv, scFv, and (Fab′)₂ fragments.

28. The method of claim 1, wherein the antibody is a bispecific antibody that recognizes a first antigen and a second antigen.

29. The method of claim 28, wherein the first antigen is selected from C1q, C1r, C1s, and the C1 complex, and the second antigen is an antigen that facilitates transport across the blood-brain-barrier.

30. The method of claim 28, wherein the second antigen is selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, or ANG1005.

31. The method of claim 1, wherein the antibody inhibits C3c deposition, membrane attack complex (MAC) deposition, axonal damage formation, or respiratory muscle damage.

32-34. (canceled)

35. The method of claim 1, wherein the antibody inhibits the classical or alternative complement activation pathway by an amount from at least 30% to at least 99.9%.

36. (canceled)

37. The method claim 1, wherein the antibody inhibits complement-dependent cell-mediated cytotoxicity (CDCC) activation pathway by an amount from at least 30% to at least 99.9%.

38. (canceled)

39. The method of claim 1, wherein the antibody has a dissociation constant (K_{D}) for its corresponding antigen from 100 nM to 0.005 nM or less than 0.005 nM.

40. The method of claim 1, wherein the antibody inhibits autoantibody-dependent and complement-dependent cytotoxicity (CDC).

41. The method of claim 1, wherein the antibody inhibits amplification of the alternative complement activation pathway initiated by C1q binding.

42-43. (canceled)

44. The method of claim 1, further comprising administering a second antibody, wherein the second antibody is selected from an anti-C1q antibody, an anti-C1r antibody, an anti-C1s antibody and an anti-C1 complex antibody.

45-48. (canceled)


SEQUENCE LISTING (12 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 4 - protein, 688 aa, Homo sapiens
MWCIVLFSLLAWVYAEPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 5 - protein, 704 aa, Homo sapiens
MWLLYLLVPALFCRAGGSIPIPQKLFGEVTSPLFPKPYPNNFETTTVITVPTGYRVKLVFQQDLEPSEGCFYDYVKISADKKSLGRFCGQLGSPLGNPPGKKEFMSQGNKMLLTFHTDFSNEENGTIMFYKGFLAYYQAVDLDECASRSKLGEEDPQPQCQHLCHNYVGGYFCSCRPGYELQEDRHSCQAECSSELYTEASGYISSLEYPRSYPPDLRCNYSIRVERGLTLHLKFLEPFDIDDHQQVHCPYDQLQIYANGKNIGEFCGKQRPPDLDTSSNAVDLLFFTDESGDSRGWKLRYTTEIIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 6 - protein, 245 aa, Mus musculus
METSQGWLVACVLTMTLVWTVAEDVCRAPNGKDGAPGNPGRPGRPGLKGERGEPGAAGIRTGIRGFKGDPGESGPPGKPGNVGLPGPSGPLGDSGPQGLKGVKGNPGNIRDQPRPAFSAIRQNPMTLGNVVIFDKVLTNQESPYQNHTGRFICAVPGFYYFNFQVISKWDLCLFIKSSSGGQPRDSLSFSNTNNKGLFQVLAGGTVLQLRRGDEVWIEKDPAKGRIYQGTEADSIFSGFLIFPSA

SEQ ID NO: 7 - protein, 246 aa, Mus musculus
MVVGPSCQPPCGLCLLLLFLLALPLRSQASAGCYGIPGMPGMPGAPGKDGHDGLQGPKGEPGIPAVPGTRGPKGQKGEPGMPGHRGKNGPRGTSGLPGDPGPRGPPGEPGVEGRYKQKHQSVFTVTRQTTQYPEANALVRFNSVVTNPQGHYNPSTGKFTCEVPGLYYFVYYTSHTANLCVHLNLNLARVASFCDHMFNSKQVSSGGVLLRLQRGDEVWLSVNDYNGMVGIEGSNSVFSGFLLFPD

SEQ ID NO: 8 - protein, 24 aa, Artificial Sequence
  Synthetic Construct
GLFQVVSGGMVLQLQQGDQVWVEK

SEQ ID NO: 9 - protein, 12 aa, Artificial Sequence
  Synthetic Construct
FQVVSGGMVLQL

SEQ ID NO: 10 - protein, 16 aa, Artificial Sequence
  Synthetic Construct
YDMVGIQGSDSVFSGF

SEQ ID NO: 11 - protein, 24 aa, Artificial Sequence
  Synthetic Construct
GLFQVVSGGMVLQLQQGDQVWVEK

SEQ ID NO: 12 - protein, 9 aa, Artificial Sequence
  Synthetic Construct
RSGVKVVTF